SEQUENCE LISTING (8 sequences)

SEQ ID NO: 1 - DNA, 129 bp, synthetic construct
  source 1..129: mol_type = other DNA
atcatggagataattaaaatgataaccatctcgcaaataaataagtattttactgttttcgtaacagttttgtaataaaaaaacctataaatattccggattattcataccgtcccaccatcgggcgcg

SEQ ID NO: 2 - DNA, 3639 bp, SARS-CoV-2
  source 1..3639: mol_type = genomic DNA
atgttcgtcttcctggtgctgctgcccctggtctccagccagtgcgtgaacctgaccactaggactcagctgcctcccgcttacaccaactcattcactcgcggtgtgtactaccctgacaaggtcttccgttcttcagtgctgcactcaactcaggacctgttcctgcccttcttctccaacgtcacctggttccacgccatccacgtgtccggcaccaacggcactaagcgcttcgacaacccagtgctgcctttcaacgacggtgtctacttcgcttcaaccgagaagtccaacatcatccgtggatggatcttcggcaccactctggacagcaagactcagtctctgctgatcgtcaacaacgccaccaacgtggtcatcaaggtgtgcgagttccagttctgtaacgacccattcctgggcgtctactaccacaagaacaacaagtcatggatggagtccgagttccgcgtctactccagcgctaacaactgcactttcgagtacgtgtcccagcctttcctgatggacctggaaggaaagcagggtaacttcaagaacctgagggagttcgtgttcaagaacatcgacggatacttcaagatctacagcaagcacaccccaatcaacctggtcagagacctgcctcagggtttctctgctctggagccactggtggacctgcctatcggcatcaacatcacccgcttccagactctgctggctctgcaccgttcctacctgactccaggcgactcttcatccggatggactgctggagctgccgcttactacgtgggctacctgcagcctaggaccttcctgctgaagtacaacgaaaacggaaccatcactgacgccgtcgactgcgctctggaccctctgtcagaaaccaagtgcactctgaagtccttcaccgtggagaagggcatctaccagacttcaaacttcagggtgcagcccaccgaatccatcgtcagattccctaacatcactaacctgtgccccttcggagaggtcttcaacgccaccagattcgcttccgtgtacgcctggaacaggaagagaatctcaaactgcgtcgctgactactccgtgctgtacaactcagcctccttcagcaccttcaagtgctacggcgtgtcaccaactaagctgaacgacctgtgcttcaccaacgtctacgccgactccttcgtgatcaggggagacgaggtcagacagatcgctcctggccagactggaaagatcgccgactacaactacaagctgcccgacgacttcaccggttgcgtcatcgcttggaacagcaacaacctggactctaaagtgggtggcaactacaactacctgtaccgcctgttccgtaagtcaaacctgaagccattcgagagggacatcagcactgaaatctaccaggccggatctaccccttgcaacggtgtcgagggcttcaactgctacttccccctgcagtcctacggtttccagccaactaacggtgtgggctaccagccttacagagtggtcgtgctgagcttcgaactgctccacgctcctgctactgtgtgcggtccaaagaagtctaccaacctggtcaagaacaagtgcgtgaacttcaacttcaacggcctgaccggaactggtgtcctgaccgagagcaacaagaagttcctgcccttccagcagttcggaagggacatcgctgacaccactgacgccgtgagagacccccagaccctggaaatcctggacatcactccatgctcattcggaggtgtctccgtgatcactccaggcaccaacacttctaaccaggtcgctgtgctgtaccaggacgtcaactgcaccgaggtccctgtggccatccacgctgaccagctgacccccacttggcgcgtgtactccaccggcagcaacgtcttccagactcgtgctggttgcctgatcggcgccgagcacgtgaacaacagctacgaatgcgacatccccatcggcgctggaatctgcgcctcttaccagacccagactaacagcccacgcagggctaggtctgtggcctctcagtcaatcatcgcttacaccatgtcactgggcgctgaaaactccgtggcctactctaacaactcaatcgccatccccaccaacttcactatcagcgtgaccactgagatcctgccagtcagcatgaccaagacttctgtggactgcactatgtacatctgcggagacagcaccgaatgctctaacctgctgctgcagtacggctctttctgcacccagctgaaccgtgctctgactggaatcgccgtggagcaggacaagaacactcaggaagtcttcgctcaggtgaagcagatctacaagaccccacctatcaaggacttcggcggattcaacttctcccagatcctgcctgacccctccaagccaagcaagcgctctttcatcgaggacctgctgttcaacaaggtcactctggccgacgctggattcatcaagcagtacggagactgcctgggtgacatcgccgctcgtgacctgatctgcgctcagaagttcaacggtctgaccgtgctgcccccactgctgactgacgaaatgatcgcccagtacactagcgccctgctggctggaaccatcacttctggttggaccttcggtgctggcgccgctctgcagatccctttcgctatgcagatggcctaccgtttcaacggaatcggtgtcacccagaacgtgctgtacgagaaccagaagctgatcgctaaccagttcaactcagccatcggaaagatccaggacagcctgagctctactgcctctgctctgggcaagctgcaggacgtcgtgaaccagaacgcccaggctctgaacaccctggtcaagcagctgtcatccaacttcggtgctatcagctctgtgctgaacgacatcctgtcccgcctggacaaggtcgaggccgaagtgcagatcgaccgcctgatcactggccgtctgcagtcactgcagacctacgtgactcagcagctgatcagggccgctgaaatcagagcctccgctaacctggccgctaccaagatgagcgagtgcgtcctgggacagtctaagcgtgtggacttctgcggcaagggataccacctgatgtcattccctcagtctgctccccacggtgtggtgttcctgcacgtcacctacgtgccagcccaggagaagaacttcaccactgcccctgctatctgccacgacggcaaggctcacttccccagggaaggtgtcttcgtgagcaacggcacccactggttcgtcactcagagaaacttctacgagccacagatcatcaccactgacaacactttcgtgtctggaaactgcgacgtcgtgatcggtatcgtcaacaacaccgtgtacgaccccctgcagccagagctggactcattcaaggaggaactggacaagtacttcaagaaccacacctcccctgacgtcgacctgggcgacatctcaggaatcaacgcttccgtcgtgaacatccagaaggagatcgaccgcctgaacgaagtggccaagaacctgaacgaaagcctgatcgacctgcaggagctgggcaagtacgaacagtacatcaagtggcct

SEQ ID NO: 3 - DNA, 765 bp, Influenza virus
  source 1..765: mol_type = genomic DNA
gccaccatgagcctgctgactgaagttgaaacctacgtgctgagcattgtgcctagcggtcctctgaaggctgagatcgcccaacgcctggaggacgtgttcgctggcaagaacactgacctggaagtgctgatggaatggctaaagactaggccaattctgagccctctgaccaagggtatcctgggtttcgtgttcaccctgaccgtgcctagcgaacgtggtctgcagcgccgtcgcttcgtgcaaaacgctctgaacggtaacggtgaccctaacaacatggacaaggctgtgaagctgtaccgtaagctgaagcgtgaaatcaccttccatggcgctaaggaaatcgctctgagctactccgccggtgctttggccagctgtatgggtctgatctacaaccgtatgggtgccgtgaccactgaggtggccttcggtctggtgtgtgctacctgtgaacagatcgccgactcacagcaccgttctcaccgtcaaatggtgaccactactaaccccctcatccgccacgagaaccgtatggtgctggcctcaaccactgccaaggctatggagcagatggccggcagcagcgagcaggccgctgaagctatggacatcgcttcccaggctcgccagatggtgcaggctatgcgcaccatcggcacccacccttcctccagcgctggtctgaaggacgacctgctggagaacctccaggcctaccagaagcgcatgggagtgcagatgcagcgtttcaagtaa

SEQ ID NO: 4 - DNA, 294 bp, Influenza virus
  source 1..294: mol_type = genomic DNA
atgagcctcttgactgaagtggagacacctatccgtaacgagtggggttgtaggtgtaacgactcctccgacccactcgttatcgctgctaacatcatcggtatcctgcacctgatcctctggattctcgaccgcctgttcttcaagtgcatctaccgccgtttcaagtacggtctgaagcgtggtccttcaacagagggtgtgccagaaagcatgcgcgaagaataccgtaaggaacagcagaacgccgtggacgttgacgacggtcacttcgtgaacatcgaactggagtaa

SEQ ID NO: 5 - DNA, 183 bp, SARS-CoV-2
  source 1..183: mol_type = genomic DNA
tggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 6 - DNA, 111 bp, Influenza virus
  source 1..111: mol_type = genomic DNA
ctgagcatctactctactgtggccagctctctggccctggctatcatggtcgctggtctgtctctgtggatgtgctcaaacggctccctgcagtgccgtatctgcatctaa

SEQ ID NO: 7 - DNA, 1548 bp, Salmonella typhimurium
  source 1..1548: mol_type = genomic DNA
atgaagttcctggttaacgtggctctggttttcatggttgtctacatctcttacatctacgccgccgctcaggtcatcaacactaacagcctgtccctcctgacacaaaacaacctgaacaagtcccagagcgccctgggcaccgctatcgaacgcctctcctccggactgcgtatcaactccgctaaggacgacgctgctggtcaggccatcgctaaccgcttcacagctaacatcaagggactgacgcaggcttcacgtaatgctaatgacggtatcagcatcgctcaaaccacagagggtgctttgaacgaaatcaacaataacctccaacgtgtgcgcgagctggctgtccagtctgctaacagcacaaacagccaaagtgacctggattccattcaggctgagattacacaacgcctgaatgaaatcgatagagttagtggtcagacccagtttaatggtgttaaagtgttggctcaggataacactctgacaatccaagttggagctaatgacggtgaaacaatcgacatcgatttgaagcagatcaacagtcagaccctgggtctggacactctgaatgtgcaacaaaaatacaaggtgtctgacaccgctgctaccgttacaggatatgctgacacaaccatcgccctggataactccacttttaaagctagtgccacaggactcggaggcactgaccaaaagattgacggagacctcaagtttgatgacacaactggcaaatactacgctaaggtgactgtgaccggcggtactggcaaggacggctactacgaggtgagtgttgacaagacaaatggagaagttactctcgctggcggtgctacatcacccctgacaggcggtctgccagccactgctaccgaagatgttaagaatgttcaagttgccaacgctgacctgaccgaggctaaggctgccctgaccgctgccggtgtcaccggcaccgcttccgttgtgaagatgagctacactgacaacaacggcaagaccatcgacggtggcctcgccgtgaaggttggcgacgactactactctgccacccagaacaaggacggaagcatctccatcaacaccactaagtacaccgctgacgacggaacctccaagacagctctcaacaagctgggtggtgctgacggcaagactgaagtggtttccatcggcggcaagacctacgctgcttccaaggccgaaggccacaacttcaaggctcagcctgacctggccgaagccgctgccaccactactgaaaaccccctccagaagatcgacgccgccctggcccaagtcgacaccctccgtagcgacctgggcgccgtccagaaccgcttcaactctgctatcaccaacctgggtaacactgtgaacaacctgacatccgctcgcagccgcatcgaggactctgactacgctacagaggtgagcaacatgtcacgcgctcaaatcctgcaacaggccggcacatccgtgctggcccaggccaaccaagtgccccagaacgttctcagcctcctgcgc

SEQ ID NO: 8 - DNA, 3750 bp, SARS-CoV-2
  source 1..3750: mol_type = genomic DNA
atgttcgtcttcctggtgctgctgcccctggtctccagccagtgcgtgaacctgaccactaggactcagctgcctcccgcttacaccaactcattcactcgcggtgtgtactaccctgacaaggtcttccgttcttcagtgctgcactcaactcaggacctgttcctgcccttcttctccaacgtcacctggttccacgccatccacgtgtccggcaccaacggcactaagcgcttcgacaacccagtgctgcctttcaacgacggtgtctacttcgcttcaaccgagaagtccaacatcatccgtggatggatcttcggcaccactctggacagcaagactcagtctctgctgatcgtcaacaacgccaccaacgtggtcatcaaggtgtgcgagttccagttctgtaacgacccattcctgggcgtctactaccacaagaacaacaagtcatggatggagtccgagttccgcgtctactccagcgctaacaactgcactttcgagtacgtgtcccagcctttcctgatggacctggaaggaaagcagggtaacttcaagaacctgagggagttcgtgttcaagaacatcgacggatacttcaagatctacagcaagcacaccccaatcaacctggtcagagacctgcctcagggtttctctgctctggagccactggtggacctgcctatcggcatcaacatcacccgcttccagactctgctggctctgcaccgttcctacctgactccaggcgactcttcatccggatggactgctggagctgccgcttactacgtgggctacctgcagcctaggaccttcctgctgaagtacaacgaaaacggaaccatcactgacgccgtcgactgcgctctggaccctctgtcagaaaccaagtgcactctgaagtccttcaccgtggagaagggcatctaccagacttcaaacttcagggtgcagcccaccgaatccatcgtcagattccctaacatcactaacctgtgccccttcggagaggtcttcaacgccaccagattcgcttccgtgtacgcctggaacaggaagagaatctcaaactgcgtcgctgactactccgtgctgtacaactcagcctccttcagcaccttcaagtgctacggcgtgtcaccaactaagctgaacgacctgtgcttcaccaacgtctacgccgactccttcgtgatcaggggagacgaggtcagacagatcgctcctggccagactggaaagatcgccgactacaactacaagctgcccgacgacttcaccggttgcgtcatcgcttggaacagcaacaacctggactctaaagtgggtggcaactacaactacctgtaccgcctgttccgtaagtcaaacctgaagccattcgagagggacatcagcactgaaatctaccaggccggatctaccccttgcaacggtgtcgagggcttcaactgctacttccccctgcagtcctacggtttccagccaactaacggtgtgggctaccagccttacagagtggtcgtgctgagcttcgaactgctccacgctcctgctactgtgtgcggtccaaagaagtctaccaacctggtcaagaacaagtgcgtgaacttcaacttcaacggcctgaccggaactggtgtcctgaccgagagcaacaagaagttcctgcccttccagcagttcggaagggacatcgctgacaccactgacgccgtgagagacccccagaccctggaaatcctggacatcactccatgctcattcggaggtgtctccgtgatcactccaggcaccaacacttctaaccaggtcgctgtgctgtaccaggacgtcaactgcaccgaggtccctgtggccatccacgctgaccagctgacccccacttggcgcgtgtactccaccggcagcaacgtcttccagactcgtgctggttgcctgatcggcgccgagcacgtgaacaacagctacgaatgcgacatccccatcggcgctggaatctgcgcctcttaccagacccagactaacagcccacgcagggctaggtctgtggcctctcagtcaatcatcgcttacaccatgtcactgggcgctgaaaactccgtggcctactctaacaactcaatcgccatccccaccaacttcactatcagcgtgaccactgagatcctgccagtcagcatgaccaagacttctgtggactgcactatgtacatctgcggagacagcaccgaatgctctaacctgctgctgcagtacggctctttctgcacccagctgaaccgtgctctgactggaatcgccgtggagcaggacaagaacactcaggaagtcttcgctcaggtgaagcagatctacaagaccccacctatcaaggacttcggcggattcaacttctcccagatcctgcctgacccctccaagccaagcaagcgctctttcatcgaggacctgctgttcaacaaggtcactctggccgacgctggattcatcaagcagtacggagactgcctgggtgacatcgccgctcgtgacctgatctgcgctcagaagttcaacggtctgaccgtgctgcccccactgctgactgacgaaatgatcgcccagtacactagcgccctgctggctggaaccatcacttctggttggaccttcggtgctggcgccgctctgcagatccctttcgctatgcagatggcctaccgtttcaacggaatcggtgtcacccagaacgtgctgtacgagaaccagaagctgatcgctaaccagttcaactcagccatcggaaagatccaggacagcctgagctctactgcctctgctctgggcaagctgcaggacgtcgtgaaccagaacgcccaggctctgaacaccctggtcaagcagctgtcatccaacttcggtgctatcagctctgtgctgaacgacatcctgtcccgcctggacaaggtcgaggccgaagtgcagatcgaccgcctgatcactggccgtctgcagtcactgcagacctacgtgactcagcagctgatcagggccgctgaaatcagagcctccgctaacctggccgctaccaagatgagcgagtgcgtcctgggacagtctaagcgtgtggacttctgcggcaagggataccacctgatgtcattccctcagtctgctccccacggtgtggtgttcctgcacgtcacctacgtgccagcccaggagaagaacttcaccactgcccctgctatctgccacgacggcaaggctcacttccccagggaaggtgtcttcgtgagcaacggcacccactggttcgtcactcagagaaacttctacgagccacagatcatcaccactgacaacactttcgtgtctggaaactgcgacgtcgtgatcggtatcgtcaacaacaccgtgtacgaccccctgcagccagagctggactcattcaaggaggaactggacaagtacttcaagaaccacacctcccctgacgtcgacctgggcgacatctcaggaatcaacgcttccgtcgtgaacatccagaaggagatcgaccgcctgaacgaagtggccaagaacctgaacgaaagcctgatcgacctgcaggagctgggcaagtacgaacagtacatcaagtggcctctgagcatctactctactgtggccagctctctggccctggctatcatggtcgctggtctgtctctgtggatgtgctcaaacggctccctgcagtgccgtatctgcatctaa

(19) United States
(12) Patent Application Publication
(10) Pub. No.: US 2023/0058789 A1
(43) Pub. Date: Feb. 23, 2023

(54) SARS-COV-2 CHIMERIC VLP VACCINE COMPOSITION, EXPRESSING VECTOR AND USE THEREOF

(71) Applicant: National Tsing Hua University, Hsinchu (TW)

(72) Inventors: SUH CHIN WU, Hsinchu (TW); Wei Shuo Lin, Hsinchu (TW); Ting Hsuan Chen, Hsinchu (TW)

(21) Appl. No.: 17/816,495

(22) Filed: Aug. 1, 2022

(30) Foreign Application Priority Data
Aug. 17, 2021 (TW) 110130372

(51) Int. Cl. C07K 14/005; A61K 39/215; A61P 31/14

(52) U.S. Cl. CPC C07K 14/005; A61K 39/215; A61P 31/14; C12N 2770/18022; C12N 2770/18023; C12N 2770/18034; C12N 2770/18071; C12N 2760/16122; C12N 2760/16134; C12N 2760/16171; A61K 2039/575

(57) ABSTRACT

The present invention provides a SARS-CoV-2 chimeric VLP vaccine composition and an expressing vector and use thereof. The chimeric SARS-CoV-2 VLP comprises a VLP skeleton formed by the M1 protein and the M2 protein of influenza virus, and the chimeric spike protein of SARS-CoV-2, expressed on the surface of the VLP skeleton, the transmembrane domain of which is replaced by the transmembrane domain of a HA of influenza virus. The present invention also provides a recombinant vector expressing the chimeric SARS-CoV-2 VLP, and the use of the chimeric SARS-CoV-2 VLP for eliciting an immune response against SARS-CoV-2 variants.

### CROSS-REFERENCE TO RELATED APPLICATION

This application claims priority of Taiwan patent application No. 110130372, filed on Aug. 17, 2021, the content of which is incorporated herein in its entirety by reference.

### BACKGROUND OF THE INVENTION

### 1. Field of the Invention

The present invention relates to a severe acute respiratory syndrome coronavirus 2 (SARS-CoV-2) chimeric virus-like particle (VLP) vaccine composition and expressing vector and use thereof. In particular, the present invention relates to a SARS-CoV-2 chimeric VLP vaccine composition comprising the chimeric spike protein of SARS-CoV-2 expressed on the surface of the virus-like particle skeleton of influenza virus, and expressing vector thereof and use thereof.

### 2. The Prior Art

Severe acute respiratory syndrome coronavirus 2 (SARS-CoV-2) is an enveloped positive-sense single-stranded RNA virus, belonging to severe acute respiratory syndrome-related coronavirus of Betacolonavirus of the Coronaviradae family. The virus particles are round or elliptical, with a diameter of about 80-120 nanometers. The virus particles are coated by a double layer of phospholipids provided by the host cell and mainly contain four structure proteins including envelope proteins (E protein), membrane protein (M protein), nucleocapsid, and spike protein (S protein). SARS-CoV-2 caused Coronavirus disease 2019 (COVID-19) that broke out at the end of 2019. SARS-CoV-2 can invade the human cells through ACE2 receptor expressed on the surface of various cells. The main infected organs include lung, heart, kidney and many other major organs.

Currently, commonly used vaccines include inactivated vaccines and attenuated vaccines, in which external forces are utilized to inactivate epitopes or reduce toxicity from real viruses. However, there is a risk of infection after vaccination owing to incomplete removal of genetic components. Virus-like particle (VLP) is a shell structure composed of viral coat protein, which does not contain virus genome. Thus, it doesn't need to be concerned about virus infection and replication. Besides, compared with subunit vaccines, because the structure of virus-like particles is similar to real viruses, after entering the individual, virus-like particles can stimulate the immune responses to produce specific antibodies to resist the invasion of the real viruses, thereby achieving a good protective effect. Therefore, virus-like particle is a form of vaccine with excellent potential.

However, virus-like particles require the combination of adjuvants to effectively enhance the immune responses of vaccinated individuals. In addition, all kinds of COVID-19 vaccines currently on the market or in clinical trials are designed for the ancestral Wuhan strain, and provide reduced protection against emerging variants. Therefore, in response to the future viral mutations, it is necessary to develop vaccine compositions that can be flexibly modified antigens in order to effectively reduce the cost and time of vaccine development, and in the same time, maintain effective and safe immune-induced responses induced by the vaccine compositions.

### SUMMARY OF THE INVENTION

To address the foregoing problem, one objective of the present invention is to provide a severe acute respiratory syndrome coronavirus 2 (SARS-CoV-2) chimeric virus-like particle (VLP) vaccine composition, comprising a SARS-CoV-2 chimeric VLP, wherein, the SARS-CoV-2 chimeric VLP comprises: a virus-like particle skeleton, formed by a matrix protein 1 (M1 protein) of influenza virus and a matrix protein 2 (M2 protein) of influenza virus; and a chimeric spike protein of SARS-CoV-2, expressed on the surface of the virus-like particle skeleton; wherein the chimeric spike protein of SARS-CoV-2 is a spike protein of SARS-CoV-2 the transmembrane domain of which is replaced by a hemagglutinin (HA) of influenza virus.

In one embodiment of the present invention, an N-terminal end of the M2 protein of influenza virus is further connected to a flagellin.

In one embodiment of the present invention, a flexible polyglycine linker is included between the M2 protein of influenza virus and the flagellin.

In one embodiment of the present invention, the SARS-CoV-2 chimeric VLP further comprises a HA of influenza virus expressed on the surface of the virus-like particle skeleton, forming a chimeric bivalent VLP of SARS-CoV-2 and influenza virus.

In one embodiment of the present invention, the chimeric bivalent VLP of SARS-CoV-2 and influenza virus is used as a bivalent vaccine composition against SARS-CoV-2 and influenza virus.

Another objective of the present invention is to provide a method of the SARS-CoV-2 chimeric VLP vaccine composition for eliciting an immune response against one or more SARS-CoV-2 variants infection, comprising immunizing a subject in need thereof a therapeutically effective amount of the SARS-CoV-2 chimeric VLP vaccine composition, which comprises a SARS-CoV-2 chimeric VLP, wherein the SARS-CoV-2 chimeric VLP comprises: a virus-like particle skeleton, formed by a M1 protein of influenza virus and a M2 protein of influenza virus; and a chimeric spike protein of SARS-CoV-2, expressed on the surface of the virus-like particle skeleton, the transmembrane domain of which is replaced by a HA of influenza virus.

In one embodiment of the present invention, the vaccine composition elicits an immune response of type I helper T cells.

In one embodiment of the present invention, the immune response of type I helper T cells includes a secretion of IgG2a, INF-γ, or the combination thereof.

In one embodiment of the present invention, the vaccine composition elicits high titer neutralizing antibodies against SARS-CoV-2.

In one embodiment of the present invention, the SARS-CoV-2 variants infection is caused by an ancestral Wuhan-Hu-1 strain, an Alpha (B.1.1.7, British) variant strain, a Beta (B.1351, South African) variant strain, a Delta (B.1.617.2, Indian) variant strain, and any combination thereof.

In one embodiment of the present invention, the SARS-CoV-2 chimeric VLP further comprises a HA of influenza virus expressed on the surface of the virus-like particle skeleton, forming a chimeric bivalent VLP of SARS-CoV-2 and influenza virus.

The other objective of the present invention is to provide a recombinant vector expressing a SARS-CoV-2 chimeric VLP, comprising: a M1 protein gene of influenza virus, a M2 protein gene of influenza virus, and a chimeric spike protein gene of SARS-CoV-2; wherein the chimeric spike protein gene of SARS-CoV-2 is a spike protein gene of SARS-CoV-2 the transmembrane domain of which is replaced by a transmembrane domain of a HA of influenza virus.

In one embodiment of the present invention, each of the M1 protein gene of influenza virus, the M2 protein gene of influenza virus, and the chimeric spike protein gene of SARS-CoV-2 is controlled by its own promoter and terminator and flanked by a pair of restriction enzyme cutting sites.

In one embodiment of the present invention, a 5′-end of the M2 protein gene of influenza virus is connected to a flagellin gene.

In one embodiment of the present invention, the recombinant vector further comprises a HA gene of influenza virus.

The SARS-CoV-2 chimeric VLP of the present invention is successfully assembled into a virus-like particle expressing a spike protein, M1 protein, and M2 protein/flagellin fusion protein constructed on a single vector of the present invention. The SARS-CoV-2 chimeric VLP of the present invention can effectively elicit the production of antibodies against SARS-CoV-2 without additional adjuvants in vaccinated individuals. Further analyses show that the amount of serum IgG2a antibodies against spike protein and the secretion of IFN-γ would increase accordingly as the dosage of the SARS-CoV-2 chimeric VLP of the present invention increases. The results indicate that the SARS-CoV-2 chimeric VLP of the present invention tends to elicit immune responses of the type I helper T cells, and could decrease the incidence of the adverse effects caused by the type II helper T cells. Furthermore, the SARS-CoV-2 chimeric VLP of the present invention can also produce high-titer neutralizing antibodies against not only the ancestral Wuhan-Hu-1 strain, but also the variant strains of Alpha (B.1.1.7, British), Beta (B.1351, South African), and Delta (B.1.617.2, Indian). As mentioned above, the SARS-CoV-2 chimeric VLP of the present invention can be effectively used as a vaccine against SARS-CoV-2.

In the SARS-CoV-2 chimeric VLP of the present invention, the transmembrane domain of the spike protein of SARS-CoV-2 is replaced with the transmembrane domain of the hemagglutinin of influenza virus to form a chimeric spike protein, which can be stably expressed on the surface of the virus-like particle skeleton. Because the SARS-CoV-2 chimeric VLP of the present invention is self-carrying flagellin proteins as an adjuvant, the SARS-CoV-2 chimeric VLP of the present invention can overcome the problem that the conventional virus-like particles still need to be used with an adjuvant.

Furthermore, in the vector for expressing the SARS-CoV-2 chimeric VLP of the present invention, all genes are under the control of their own promoter and terminator, so each of them can be expressed independently to reduce the risk of mutual interference. Moreover, different restriction enzyme cutting sites are designed on upstream and downstream of each gene above. The proteins exhibited on the SARS-CoV-2 chimeric VLP of the present invention can be easily replaced, accelerating the vaccine development when the evolving mutations of SARS-CoV-2 emerge. In this way, the present invention provides a new vaccine platform that does not require additional adjuvants and can be modified with antigens flexibly.

The embodiments of the present invention are further described with the following drawings. The following embodiments are given to illustrate the present invention and are not intended to limit the scope of the present invention, and those having ordinary skill in the art can make some modifications and refinements without departing from the spirit and scope of the present invention. Therefore, the scope of the present invention is defined by the scope of the appended claims.

### BRIEF DESCRIPTION OF THE DRAWINGS

FIG. 1A shows a schematic view of the SARS-CoV-2 chimeric VLP according to one embodiment of the present invention.

FIG. 1B shows schematic view of the design of vector for expressing the SARS-CoV-2 chimeric VLP according to one embodiment of the present invention; wherein, po1H represents polyhedrin promoter (SEQ ID NO:1); S-HA™ represents the spike protein gene of SARS-CoV-2, and the Transmembrane Domain™ thereof is replaced with the transmembrane domain of hemagglutinin (HA) of influenza virus; M1 represents M1 protein gene; FliC-M2 represents M2 protein/flagellin fusion protein gene; furthermore, BamHI, EcoR1, Mlu1, Kpn1, Xho1, and HindIII represent cutting sites of different restrictions enzyme.

FIG. 2A shows a result of western blot assay of each fraction of the SARS-CoV-2 chimeric VLP of the present invention purified by sucrose gradients; wherein, the numbers 1 to 12 represent continuous sucrose concentration interval solutions.

FIG. 2B shows an image of the appearance of the SARS-CoV-2 chimeric VLP of the present invention under transmission electron microscope (TEM).

FIG. 2C shows a result of quantifying the spike protein content in the SARS-CoV-2 chimeric VLP of the present invention by analyzing the results of the western blot assay; wherein, S represents spike protein.

FIG. 3A shows titers of anti-spike protein antibodies in the serum of mice immunized with the SARS-CoV-2 chimeric VLP of the present invention by enzyme-linked immunosorbent assay (ELISA); wherein, PBS represents the serum of control group of mice immunized with PBS solution only; 2 μg S FliC-cVLP represents the serum of experimental group of mice immunized with 2 μg of the SARS-CoV-2 chimeric VLP of the present invention; 2 μg S FliC-cVLP+Alum represents the serum of comparison group of mice immunized with 2 μg of the SARS-CoV-2 chimeric VLP of the present invention and 300 μg of alum; 2 μg S FliC-cVLP+MPLA represents the serum of comparison group of mice immunized with 2 μg of the SARS-CoV-2 chimeric VLP of the present invention and 30 μg of MPLA (monophosphoryl lipid A); 2 μg S FliC-cVLP+Alum+MPLA represents the serum of comparison group of mice immunized with 2 μg of the SARS-CoV-2 chimeric VLP of the present invention, 300 μg of alum, and 30 μg of MPLA; 20 μg S FliC-cVLP represents the serum of experimental group of mice immunized with 20 μg of the SARS-CoV-2 chimeric VLP of the present invention; 20 μg S FliC-cVLP+Alum represents the serum of comparison group of mice immunized with 20 μg of the SARS-CoV-2 chimeric VLP of the present invention and 300 μg of alum; 20 μg S FliC-cVLP+MPLA represents the serum of comparison group of mice immunized with 20 μg of the SARS-CoV-2 chimeric VLP of the present invention and 30 μg of MPLA (monophosphoryl lipid A); 20 μg S FliC-cVLP+Alum+MPLA represents the serum of comparison group of mice immunized with 20 μg of the SARS-CoV-2 chimeric VLP of the present invention, 300 μg of alum, and 30 μg of MPLA; 40 μg S FliC-cVLP represents the serum of experimental group of mice immunized with 40 μg of the SARS-CoV-2 chimeric VLP of the present invention; 40 μg S FliC-cVLP+MPLA represents the serum of comparison group of mice immunized with 40 μg of the SARS-CoV-2 chimeric VLP of the present invention and 30 μg of MPLA; 80 μg S FliC-cVLP represents the serum of experimental group of mice immunized with 80 μg of the SARS-CoV-2 chimeric VLP of the present invention; 80 μg S FliC-cVLP+MPLA represents the serum of comparison group of mice immunized with 80 μg of the SARS-CoV-2 chimeric VLP of the present invention and 30 μg of MPLA.

FIG. 3B shows titers of anti-RBD (receptor binding domain) antibodies in the serum of mice immunized with the SARS-CoV-2 chimeric VLP of the present invention by enzyme-linked immunosorbent assay (ELISA); wherein, PBS represents the serum of control group of mice immunized with PBS solution only; 2 μg S FliC-cVLP represents the serum of experimental group of mice immunized with 2 μg of the SARS-CoV-2 chimeric VLP of the present invention; 2 μg S FliC-cVLP+Alum represents the serum of comparison group of mice immunized with 2 μg of the SARS-CoV-2 chimeric VLP of the present invention and 300 μg of alum; 2 μg S FliC-cVLP+MPLA represents the serum of comparison group of mice immunized with 2 μg of the SARS-CoV-2 chimeric VLP of the present invention and 30 μg of MPLA (monophosphoryl lipid A); 2 μg S FliC-cVLP+Alum+MPLA represents the serum of comparison group of mice immunized with 2 μg of the SARS-CoV-2 chimeric VLP of the present invention, 300 μg of alum, and 30 μg of MPLA; 20 μg S FliC-cVLP represents the serum of experimental group of mice immunized with 20 μg of the SARS-CoV-2 chimeric VLP of the present invention; 20 μg S FliC-cVLP+Alum represents the serum of comparison group of mice immunized with 20 μg of the SARS-CoV-2 chimeric VLP of the present invention and 300 μg of alum; 20 μg S FliC-cVLP+MPLA represents the serum of comparison group of mice immunized with 20 μg of the SARS-CoV-2 chimeric VLP of the present invention and 30 μg of MPLA (monophosphoryl lipid A); 20 μg S FliC-cVLP+Alum+MPLA represents the serum of comparison group of mice immunized with 20 μg of the SARS-CoV-2 chimeric VLP of the present invention, 300 μg of alum, and 30 μg of MPLA; 40 μg S FliC-cVLP represents the serum of experimental group of mice immunized with 40 μg of the SARS-CoV-2 chimeric VLP of the present invention; 40 μg S FliC-cVLP+MPLA represents the serum of comparison group of mice immunized with 40 μg of the SARS-CoV-2 chimeric VLP of the present invention and 30 μg of MPLA; 80 μg S FliC-cVLP represents the serum of experimental group of mice immunized with 80 μg of the SARS-CoV-2 chimeric VLP of the present invention; 80 μg S FliC-cVLP+MPLA represents the serum of comparison group of mice immunized with 80 μg of the SARS-CoV-2 chimeric VLP of the present invention and 30 μg of MPLA.

FIG. 4A shows titers of anti-spike protein IgG2a antibodies in the serum of mice immunized with the SARS-CoV-2 chimeric VLP of the present invention by enzyme-linked immunosorbent assay (ELISA); wherein, PBS represents the serum of control group of mice immunized with PBS solution only; 2 μg S FliC-cVLP represents the serum of experimental group of mice immunized with 2 μg of the SARS-CoV-2 chimeric VLP of the present invention; 2 μg S FliC-cVLP+Alum represents the serum of comparison group of mice immunized with 2 μg of the SARS-CoV-2 chimeric VLP of the present invention and 300 μg of alum; 2 μg S FliC-cVLP+MPLA represents the serum of comparison group of mice immunized with 2 μg of the SARS-CoV-2 chimeric VLP of the present invention and 30 μg of MPLA (monophosphoryl lipid A); 2 μg S FliC-cVLP+Alum+MPLA represents the serum of comparison group of mice immunized with 2 μg of the SARS-CoV-2 chimeric VLP of the present invention, 300 μg of alum, and 30 μg of MPLA; 20 μg S FliC-cVLP represents the serum of experimental group of mice immunized with 20 μg of the SARS-CoV-2 chimeric VLP of the present invention; 20 μg S FliC-cVLP+Alum represents the serum of comparison group of mice immunized with 20 μg of the SARS-CoV-2 chimeric VLP of the present invention and 300 μg of alum; 20 μg S FliC-cVLP+MPLA represents the serum of comparison group of mice immunized with 20 μg of the SARS-CoV-2 chimeric VLP of the present invention and 30 μg of MPLA (monophosphoryl lipid A); 20 μg S FliC-cVLP+Alum+MPLA represents the serum of comparison group of mice immunized with 20 μg of the SARS-CoV-2 chimeric VLP of the present invention, 300 μg of alum, and 30 μg of MPLA; 40 μg S FliC-cVLP represents the serum of experimental group of mice immunized with 40 μg of the SARS-CoV-2 chimeric VLP of the present invention; 40 μg S FliC-cVLP+MPLA represents the serum of comparison group of mice immunized with 40 μg of the SARS-CoV-2 chimeric VLP of the present invention and 30 μg of MPLA; 80 μg S FliC-cVLP represents the serum of experimental group of mice immunized with 80 μg of the SARS-CoV-2 chimeric VLP of the present invention; 80 μg S FliC-cVLP+MPLA represents the serum of comparison group of mice immunized with 80 μg of the SARS-CoV-2 chimeric VLP of the present invention and 30 μg of MPLA.

FIG. 4B shows titers of anti-spike protein IgG1 antibodies in the serum of mice immunized with the SARS-CoV-2 chimeric VLP of the present invention by enzyme-linked immunosorbent assay (ELISA); wherein, PBS represents the serum of control group of mice immunized with PBS solution only; 2 μg S FliC-cVLP represents the serum of experimental group of mice immunized with 2 μg of the SARS-CoV-2 chimeric VLP of the present invention; 2 μg S FliC-cVLP+Alum represents the serum of comparison group of mice immunized with 2 μg of the SARS-CoV-2 chimeric VLP of the present invention and 300 μg of alum; 2 μg S FliC-cVLP+MPLA represents the serum of comparison group of mice immunized with 2 μg of the SARS-CoV-2 chimeric VLP of the present invention and 30 μg of MPLA (monophosphoryl lipid A); 2 μg S FliC-cVLP+Alum+MPLA represents the serum of comparison group of mice immunized with 2 μg of the SARS-CoV-2 chimeric VLP of the present invention, 300 μg of alum, and 30 μg of MPLA; 20 μg S FliC-cVLP represents the serum of experimental group of mice immunized with 20 μg of the SARS-CoV-2 chimeric VLP of the present invention; 20 μg S FliC-cVLP+Alum represents the serum of comparison group of mice immunized with 20 μg of the SARS-CoV-2 chimeric VLP of the present invention and 300 μg of alum; 20 μg S FliC-cVLP+MPLA represents the serum of comparison group of mice immunized with 20 μg of the SARS-CoV-2 chimeric VLP of the present invention and 30 μg of MPLA (monophosphoryl lipid A); 20 μg S FliC-cVLP+Alum+MPLA represents the serum of comparison group of mice immunized with 20 μg of the SARS-CoV-2 chimeric VLP of the present invention, 300 μg of alum, and 30 μg of MPLA; 40 μg S FliC-cVLP represents the serum of experimental group of mice immunized with 40 μg of the SARS-CoV-2 chimeric VLP of the present invention; 40 μg S FliC-cVLP+MPLA represents the serum of comparison group of mice immunized with 40 μg of the SARS-CoV-2 chimeric VLP of the present invention and 30 μg of MPLA; 80 μg S FliC-cVLP represents the serum of experimental group of mice immunized with 80 μg of the SARS-CoV-2 chimeric VLP of the present invention; 80 μg S FliC-cVLP+MPLA represents the serum of comparison group of mice immunized with 80 μg of the SARS-CoV-2 chimeric VLP of the present invention and 30 μg of MPLA.

FIG. 4C shows the concentration of IFN-γ produced by spleen cells of mice immunized with the SARS-CoV-2 chimeric VLP of the present invention and then stimulated with spike protein by enzyme-linked immunosorbent assay (ELISA); wherein, PBS represents the spleen cell of control group of mice immunized with PBS solution only; 2 μg S FliC-cVLP represents the spleen cell of experimental group of mice immunized with 2 μg of the SARS-CoV-2 chimeric VLP of the present invention; 2 μg S FliC-cVLP+Alum represents the spleen cell of comparison group of mice immunized with 2 μg of the SARS-CoV-2 chimeric VLP of the present invention and 300 μg of alum; 2 μg S FliC-cVLP+MPLA represents the spleen cell of comparison group of mice immunized with 2 μg of the SARS-CoV-2 chimeric VLP of the present invention and 30 μg of MPLA (monophosphoryl lipid A); 2 μg S FliC-cVLP+Alum+MPLA represents the spleen cell of comparison group of mice immunized with 2 μg of the SARS-CoV-2 chimeric VLP of the present invention, 300 μg of alum, and 30 μg of MPLA; 20 μg S FliC-cVLP represents the spleen cell of experimental group of mice immunized with 20 μg of the SARS-CoV-2 chimeric VLP of the present invention; 20 μg S FliC-cVLP+Alum represents the spleen cell of comparison group of mice immunized with 20 μg of the SARS-CoV-2 chimeric VLP of the present invention and 300 μg of alum; 20 μg S FliC-cVLP+MPLA represents the spleen cell of comparison group of mice immunized with 20 μg of the SARS-CoV-2 chimeric VLP of the present invention and 30 μg of MPLA (monophosphoryl lipid A); 20 μg S FliC-cVLP+Alum+MPLA represents the spleen cell of comparison group of mice immunized with 20 μg of the SARS-CoV-2 chimeric VLP of the present invention, 300 μg of alum, and 30 μg of MPLA; 40 μg S FliC-cVLP represents the spleen cell of experimental group of mice immunized with 40 μg of the SARS-CoV-2 chimeric VLP of the present invention; 40 μg S FliC-cVLP+MPLA represents the spleen cell of comparison group of mice immunized with 40 μg of the SARS-CoV-2 chimeric VLP of the present invention and 30 μg of MPLA; 80 μg S FliC-cVLP represents the spleen cell of experimental group of mice immunized with 80 μg of the SARS-CoV-2 chimeric VLP of the present invention; 80 μg S FliC-cVLP+MPLA represents the spleen cell of comparison group of mice immunized with 80 μg of the SARS-CoV-2 chimeric VLP of the present invention and 30 μg of MPLA.

FIG. 4D shows the concentration of IL-5 produced by spleen cells of mice immunized with the SARS-CoV-2 chimeric VLP of the present invention and then stimulated with spike protein by enzyme-linked immunosorbent assay (ELISA); wherein, PBS represents the spleen cell of control group of mice immunized with PBS solution only; 2 μg S FliC-cVLP represents the spleen cell of experimental group of mice immunized with 2 μg of the SARS-CoV-2 chimeric VLP of the present invention; 2 μg S FliC-cVLP+Alum represents the spleen cell of comparison group of mice immunized with 2 μg of the SARS-CoV-2 chimeric VLP of the present invention and 300 μg of alum; 2 μg S FliC-cVLP+MPLA represents the spleen cell of comparison group of mice immunized with 2 μg of the SARS-CoV-2 chimeric VLP of the present invention and 30 μg of MPLA (monophosphoryl lipid A); 2 μg S FliC-cVLP+Alum+MPLA represents the spleen cell of comparison group of mice immunized with 2 μg of the SARS-CoV-2 chimeric VLP of the present invention, 300 μg of alum, and 30 μg of MPLA; 20 μg S FliC-cVLP represents the spleen cell of experimental group of mice immunized with 20 μg of the SARS-CoV-2 chimeric VLP of the present invention; 20 μg S FliC-cVLP+Alum represents the spleen cell of comparison group of mice immunized with 20 μg of the SARS-CoV-2 chimeric VLP of the present invention and 300 μg of alum; 20 μg S FliC-cVLP+MPLA represents the spleen cell of comparison group of mice immunized with 20 μg of the SARS-CoV-2 chimeric VLP of the present invention and 30 μg of MPLA (monophosphoryl lipid A); 20 μg S FliC-cVLP+Alum+MPLA represents the spleen cell of comparison group of mice immunized with 20 μg of the SARS-CoV-2 chimeric VLP of the present invention, 300 μg of alum, and 30 μg of MPLA; 40 μg S FliC-cVLP represents the spleen cell of experimental group of mice immunized with 40 μg of the SARS-CoV-2 chimeric VLP of the present invention; 40 μg S FliC-cVLP+MPLA represents the spleen cell of comparison group of mice immunized with 40 μg of the SARS-CoV-2 chimeric VLP of the present invention and 30 μg of MPLA; 80 μg S FliC-cVLP represents the spleen cell of experimental group of mice immunized with 80 μg of the SARS-CoV-2 chimeric VLP of the present invention; 80 μg S FliC-cVLP+MPLA represents the spleen cell of comparison group of mice immunized with 80 μg of the SARS-CoV-2 chimeric VLP of the present invention and 30 μg of MPLA.

FIG. 5A shows the neutralizing result of the serum of mice immunized with the SARS-CoV-2 chimeric VLP of the present invention against the ancestral Wuhan-Hu-1 strain of SARS-CoV-2 by pseudotyped lentivirus neutralization assay; wherein, PBS represents the serum of control group of mice immunized with PBS solution only; 40 μg S FliC-cVLP represents the serum of experimental group of mice immunized with 40 μg of the SARS-CoV-2 chimeric VLP of the present invention; 40 μg S FliC-cVLP+MPLA represents the serum of comparison group of mice immunized with 40 μg of the SARS-CoV-2 chimeric VLP of the present invention and 30 μg of MPLA; 80 μg S FliC-cVLP represents the serum of experimental group of mice immunized with 80 μg of the SARS-CoV-2 chimeric VLP of the present invention; 80 μg S FliC-cVLP+MPLA represents the serum of comparison group of mice immunized with 80 μg of the SARS-CoV-2 chimeric VLP of the present invention and 30 μg of MPLA.

FIG. 5B shows the neutralizing result of the serum of mice immunized with the SARS-CoV-2 chimeric VLP of the present invention against the Alpha (B.1.1.7, British) variant strain of SARS-CoV-2 by pseudotyped lentivirus neutralization assay; wherein, PBS represents the serum of control group of mice immunized with PBS solution only; 40 μg S FliC-cVLP represents the serum of experimental group of mice immunized with 40 μg of the SARS-CoV-2 chimeric VLP of the present invention; 40 μg S FliC-cVLP+MPLA represents the serum of comparison group of mice immunized with 40 μg of the SARS-CoV-2 chimeric VLP of the present invention and 30 μg of MPLA; 80 μg S FliC-cVLP represents the serum of experimental group of mice immunized with 80 μg of the SARS-CoV-2 chimeric VLP of the present invention; 80 μg S FliC-cVLP+MPLA represents the serum of comparison group of mice immunized with 80 μg of the SARS-CoV-2 chimeric VLP of the present invention and 30 μg of MPLA.

FIG. 5C shows the neutralizing result of the serum of mice immunized with the SARS-CoV-2 chimeric VLP of the present invention against the Beta (B.1351, South African) variant strain of SARS-CoV-2 by pseudotyped lentivirus neutralization assay; wherein, PBS represents the serum of control group of mice immunized with PBS solution only; 40 μg S FliC-cVLP represents the serum of experimental group of mice immunized with 40 μg of the SARS-CoV-2 chimeric VLP of the present invention; 40 μg S FliC-cVLP+MPLA represents the serum of comparison group of mice immunized with 40 μg of the SARS-CoV-2 chimeric VLP of the present invention and 30 μg of MPLA; 80 μg S FliC-cVLP represents the serum of experimental group of mice immunized with 80 μg of the SARS-CoV-2 chimeric VLP of the present invention; 80 μg S FliC-cVLP+MPLA represents the serum of comparison group of mice immunized with 80 μg of the SARS-CoV-2 chimeric VLP of the present invention and 30 μg of MPLA.

FIG. 5D shows the neutralizing result of the serum of mice immunized with the SARS-CoV-2 chimeric VLP of the present invention against the Delta (B.1.617.2, Indian) variant strain of SARS-CoV-2 by pseudotyped lentivirus neutralization assay; wherein, PBS represents the serum of control group of mice immunized with PBS solution only; 40 μg S FliC-cVLP represents the serum of experimental group of mice immunized with 40 μg of the SARS-CoV-2 chimeric VLP of the present invention; 40 μg S FliC-cVLP+MPLA represents the serum of comparison group of mice immunized with 40 μg of the SARS-CoV-2 chimeric VLP of the present invention and 30 μg of MPLA; 80 μg S FliC-cVLP represents the serum of experimental group of mice immunized with 80 μg of the SARS-CoV-2 chimeric VLP of the present invention; 80 μg S FliC-cVLP+MPLA represents the serum of comparison group of mice immunized with 80 μg of the SARS-CoV-2 chimeric VLP of the present invention and 30 μg of MPLA.

FIG. 5E shows IC50 of the neutralizing result of the serum of mice immunized with the SARS-CoV-2 chimeric VLP of the present invention against the ancestral Wuhan-Hu-1 strain, the Alpha variant strain, the Beta variant strain, and the Delta variant strain of SARS-CoV-2 by pseudotyped lentivirus neutralization assay; wherein, PBS represents the serum of control group of mice immunized with PBS solution only; 40 μg S FliC-cVLP represents the serum of experimental group of mice immunized with 40 μg of the SARS-CoV-2 chimeric VLP of the present invention; 40 μg S FliC-cVLP+MPLA represents the serum of comparison group of mice immunized with 40 μg of the SARS-CoV-2 chimeric VLP of the present invention and 30 μg of MPLA; 80 μg S FliC-cVLP represents the serum of experimental group of mice immunized with 80 μg of the SARS-CoV-2 chimeric VLP of the present invention; 80 μg S FliC-cVLP+MPLA represents the serum of comparison group of mice immunized with 80 μg of the SARS-CoV-2 chimeric VLP of the present invention and 30 μg of MPLA.

FIG. 6A shows a schematic view of the chimeric bivalent virus-like particle of SARS-CoV-2 and influenza virus according to one embodiment of the present invention.

FIG. 6B shows a result of western blot assay of each purified solution after purification of the chimeric bivalent virus-like particle of SARS-CoV-2 and influenza virus of the present invention purified by a sucrose gradient; wherein, the numbers 1 to 12 represent continuous sucrose concentration interval solutions.

FIG. 6C shows an image of the appearance of the chimeric bivalent virus-like particle of SARS-CoV-2 and influenza virus of the present invention under transmission electron microscope (TEM).

### DETAILED DESCRIPTION OF THE PREFERRED EMBODIMENT

All results were analyzed by GraphPad Prism v6.01. The titers of anti-spike protein antibodies and anti-receptor binding domain protein antibodies were analyzed by two-way ANOVA; and when analyzing the titers of IgG1 antibody, the titers of IgG2a antibody, the concentration of IFN-γ, and the concentration of IL-5, Dunn's multiple comparisons test was used for the low-dose (i.e., 2 μg and 20 μg) groups, and Mann-Whitney U-test was used for the high-dose (40 μg and 80 μg) groups. Statistical significance in all results is expressed as follows: *p<0.05; **p<0.01; and ***p<0.001. All experiments were performed at least twice.

### Definition

Herein, the data provided represent experimental values can vary within a range of 20%, preferably within ±10%, and most preferably within ±5%.

According to the present invention, the operating procedures and parameter conditions of gene cloning are within the professional literacy and routine techniques of those having ordinary skill in the art.

In the present invention, the matrix protein 1 of influenza virus, the matrix protein 2 of influenza virus, and the hemagglutinin of influenza virus of the SARS-CoV-2 chimeric VLP and the hemagglutinin of influenza virus of the chimeric bivalent virus-like particle of SARS-CoV-2 and influenza virus can be from the same or different influenza virus strains.

### Sodium Dodecyl Sulfate Polyacrylamide Gel Electrophoresis (SDS-PAGE)

The operation of SDS-PAGE is briefly described as follows. First, protein samples and SDS loading buffer (containing 50 mM Tris-HCl, pH 6.8; 100 mM dithiothreitol (DTT); 2% SDS; 0.1% bromophenol blue; and 10% glycerin) are mixed in a ratio of 3:1 and then are boiled for 10 minutes.

At the same time, an electrophoresis gel, which comprises: a separating gel (taking a 12% separating gel as an example: containing 2.5 mL of 1 M Tris, pH 8.8; 3.3 mL of deionized water; 4 mL of 30% acrylamide mix; 0.1 mL of 10% SDS; 0.1 mL of 10% ammonium persulfate (APS); and 0.01 mL of tetramethylethylenediamine (TEMED)); and a stacking gel (taking a 5% stacking gel as an example: containing 0.63 mL of 1 M Tris, pH 6.8; 3.4 mL of deionized water; 0.83 mL of 30% acrylamide mix; 0.05 mL of 10% SDS; 0.05 mL of 10% APS; and 0.005 mL TEMED), is prepared.

Protein focusing within the stacking gel is performed at 80V, and protein separating within the separating gel is performed at 140V; wherein, the time of electrophoresis depends on the molecular weight of loaded proteins. The electrophoresis gel is then dyed with coomassie brilliant blue dye (containing 0.1% coomassie R250; 10% acetic acid; and 50% methanol) for 1 hour, and then is decolorized with a decolorizing solution (containing 10% acetic acid; and 50% methanol).

### Western Blotting Assay

The operation of western blotting assay is briefly described as follows. In a transfer tank, the protein samples separated by SDS-PAGE on the electrophoresis gel are transferred onto a nitrocellulose membrane (NC membrane) at 135V, and then the NC membrane containing the transferred proteins is soaked in 20 mL tris-buffered saline solution containing tween-20 with 5% skimmed milk (hereinafter referred to as TBST solution, containing 50 mM Tris; 150 mM sodium chloride; and 0.05% Tween-20), and then is shaken for at least 1 hour to block non-specific binding.

The NC membrane is then washed 3 times with TBST solution, and added the primary antibody diluted with TBST solution at a specific multiple, and shaken at 4° C. for about 16 hours. After being washed 3 times with TBST solution, the NC membrane is then added the secondary antibody, which is connected to horseradish peroxidase (HRP), diluted with TBST solution at a specific multiple, and shaken at room temperature for 1 hour. After being washed 3 times with TBST solution, the NC membrane is then added with a western Lighting Plus ECL (PerkinElmer) to generate a luminescence signal, and a film (Medical X-ray Film, Fujifilm) is used to developed the luminescence signal.

### Example 1

### The Design and Construction of the Vector Expressing the SARS-CoV-2 Chimeric VLP

One embodiment of the present invention is the design and construction of the SARS-CoV-2 chimeric VLP vaccine composition of the present invention, which is embedded with flagellin protein as a self-carrying adjuvant. A spike protein (also known as S protein) of SARS-CoV-2 is expressed on the surface of virus-like particle skeleton of influenza virus to form the SARS-CoV-2 chimeric VLP vaccine composition of the present invention. In order to overcome the problem that adjuvants are still needed when using virus-like particles as vaccines, flagellin is self-carried onto the virus-like particles to serve as an adjuvant, effectively enhancing the ability to induce immune responses in vaccinated subjects.

In the example, in order to stably express the spike protein of SARS-CoV-2 on the surface of virus-like particle skeleton of influenza virus, the transmembrane domain of the spike protein is replaced with the transmembrane domain of the hemagglutinin (HA) of H5 influenza virus to form a chimeric spike protein. The gene of the chimeric spike protein and the genes of two structural proteins, i.e. a matrix protein 1 (M1 protein) and a matrix protein 2 (M2 protein), which are necessary to form influenza virus particles, are constructed in series in a single vector. The gene of flagellin is connected to the 5′end of the gene of M2 protein to produce a fusion protein of M2 protein/flagellin protein, so that the flagellin can be expressed on the virus-like particle of the present invention and act as an adjuvant.

In the example, the chimeric spike protein gene, the M1 protein gene, and the M2 protein/flagellin fusion protein gene all have their own promoter and terminator, so each of them can be expressed independently to reduce the risk of mutual interference. Besides, different cutting sites of different restriction enzymes are designed on upstream and downstream of each gene above, so the SARS-CoV-2 chimeric VLP of the present invention can be flexibly replaced with or added with other antigens to effectively reduce the cost and time of research and development to facilitate the need of replacement of antigens every time in response to new mutations of SARS-CoV-2 or evolution thereof into a seasonal infectious disease. As a result, the new vaccine platform of the present invention that does not require additional adjuvants and can be modified with antigens flexibly is designed.

Hereinafter, the preparation method of the SARS-CoV-2 chimeric VLP vaccine composition of the present invention, which is embedded with flagellin protein as a self-carrying adjuvant, would be described in detail. First, in order to construct a vector containing the virus-like particle of the present invention, the spike protein gene of SARS-CoV-2 obtained from GenScript (Wuhan-Hu-1 strain, accession number is MN908947.3), which has been codon-optimized for insect cells (SEQ ID NO. 2), M1 protein gene (SEQ ID NO. 3) of influenza virus (A/WSN/1933 (H1N1), accession number L25818.1), and M2 protein gene (SEQ ID NO. 4) of influenza virus (A/WSN/1933 (H1N1), accession number L25818.1) are cloned into a pFastBac1 vector between the restriction enzyme cutting sites of BamHI and HindIII (purchased from Invitrogen, USA); wherein, after the sequences of the transmembrane domain of the spike protein and the hemagglutinin are confirmed by online database (such as UniProt), the artificial sequence, in which the gene sequence of the transmembrane domain of the spike protein (SEQ ID NO. 5) is replaced with the gene sequence of the transmembrane domain of HA (SEQ ID NO. 6) of influenza virus (A/Thailand/1(KAN-1)/2004(H5N1)), is chemically synthesized through commercial outsource (such as Bio Basic Inc., Canada); and gene sequence of six glycine is used to ligate flagellin gene (SEQ ID NO. 7) onto the 5′end of the M2 protein gene, that is, the N-terminal end of the M2 protein would be fused with flagellin.

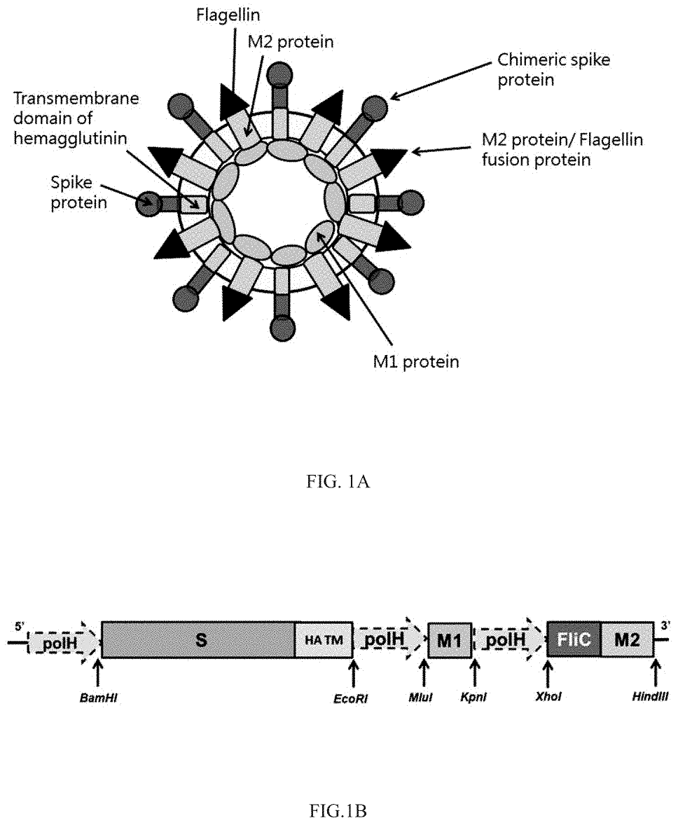

Please refer to FIGS. 1A and 1B, which are respectively a schematic view of the SARS-CoV-2 chimeric VLP according to one embodiment of the present invention and the design thereof. As shown in FIG. 1A, the M1 protein and the M2 protein are used in the present invention to form a virus-like particle skeleton, which the M1 protein is located on the inner side and the M2 protein is located on the outer side to form a spherical structure, and the chimeric spike protein is expressed on the surface of the virus-like particle skeleton. The flagellin is fused to the outer side of the M2 protein. As shown in FIG. 1B, the present invention can be cleaved by restriction enzymes of BamHI and EcoR1 to replace the chimeric spike protein gene (SEQ ID NO. 8) (S and HA™ in FIG. 1B, S represents the spike protein gene, which does not include the gene sequence of the transmembrane domain of the spike protein; and HA™ represents the gene sequence of the transmembrane domain of hemagglutinin of influenza virus). Restriction enzymes Mlu1 and Kpn1 are utilized to replace the M1 protein gene, and Xho1 and HindIII are utilized to replace the M2 protein/flagellin (shown as FliC in FIG. 1B) fusion protein gene. The restriction enzyme cutting sites described above are only examples in the embodiment, and one with ordinary skill in the art of the present invention can be employed with other restriction enzyme.

In this way, other gene sequences of antigen proteins can be arbitrarily replaced or added onto the vector to prepare virus-like particles of the present invention with other antigens. The position of the chimeric spike protein gene (i.e., S or S plus HA™ as shown in FIG. 1B) could be replaced with other antigen proteins, especially for the emerging mutations of SARS-CoV-2, to reduce the cost and time of research and facilitate the need of replacing antigens every time in response to evolving SARS-CoV-2.

Next, in order to prepare the virus-like particles of the present invention, the Bac-to-Bac® Baculovirus Expression System (purchased from Invitrogen, USA) is used to produce a recombinant baculovirus with the vector expressing the virus-like particle of the present invention. Insect cells, such as Sf9 cells, are then infected with the recombinant baculovirus, so that the insect cells can express the proteins in the vector through the recombinant baculovirus to prepare the virus-like particles of the present invention. The detailed preparation method is as follows: first, 1 ng of the pFastBac1 vector, which is cloned with the chimeric spike protein gene, the M1 protein gene, and the M2 protein/flagellin fusion protein gene, is well mixed with 90 L of competent cells (E. coli DH10 Bac strain, purchased from Invitrogen, USA) and the mixture is put on ice for 30 minutes. In order to transform the vector into competent cells of the Bac-to-Bac® baculovirus expression system, the mixture is then put in a 42° C. environment for heat shock for 45 seconds and stand on ice for 2 minutes. 900 μL of bacterial culture medium (such as LB broth) is added into the competent cells, and then incubated in a 37° C. incubator for 4 hours. Then, 10 μL of the culture medium is taken out and spread evenly on a LB culture plate with 50 g/mL kanamycin, 7 μg/mL gentamicin, 10 μg/mL tetracycline, 100 μg/mL X-gal, and 40 μg/mL isopropyl-β-D-thiogalactin glycosides to select competent cells that the expressing vectors have been successfully transformed into. Such competent cells are cultured with conventional methods to make the vector recombine with the parental bacmids in the competent cells to form recombinant bacmid.

The expression bacmid is then purified from the competent cells, and 5 μg of the expression bacmid and 16 μL of turbofect transfection reagent (purchased from Thermo Scientific™, USA) and OPTI-MEM culture medium (purchased from Thermo Scientific™, USA) are mixed together and stand for 15 minutes until the expression bacmid and transfection reagent are completely mixed. Then the mixture is added to the adherent Sf9 cells, and stands in a 28° C. constant temperature incubator for 6 hours to prepare a recombinant baculovirus with the expression bacmid for subsequent protein expression. Next, the culture medium is exchanged with 3 mL of fresh Sf900IISFM (purchased from Thermo Scientific™, USA, containing 10% penicillin/streptomycin antibiotics) containing 5% FBS. The collected recombinant baculovirus is used to infect Sf9 cells for 120 hours, so that the Sf9 cells could express the chimeric spike protein, the M1 protein, and M2 protein/flagellin fusion protein in the vector through the recombinant baculovirus to form the SARS-CoV-2 chimeric VLP of the present invention. The Sf9 cells and culture medium thereof are collected after infection. Centrifuged at 10,000 rpm, the Sf9 cells are separated, and the supernatant, which contains the virus-like particles of the present invention, is collected. After the supernatant is filtered and concentrated with a 100 kDa filter membrane, the concentrated solution is added on the top of a discontinuous sucrose gradient (0%, 20%, 30%, 40%, 50% and 60%) to perform ultracentrifugation at 28,200 rpm for 4 hours for purification. After centrifugation, the solvent of the separated products in the concentrated solution is replaced with phosphate buffered saline (PBS) for storage, and a small number of samples of each separated product are taken to confirm which sucrose concentration range that the virus-like particles of the present invention are located in by SDS-PAGE and western blotting.

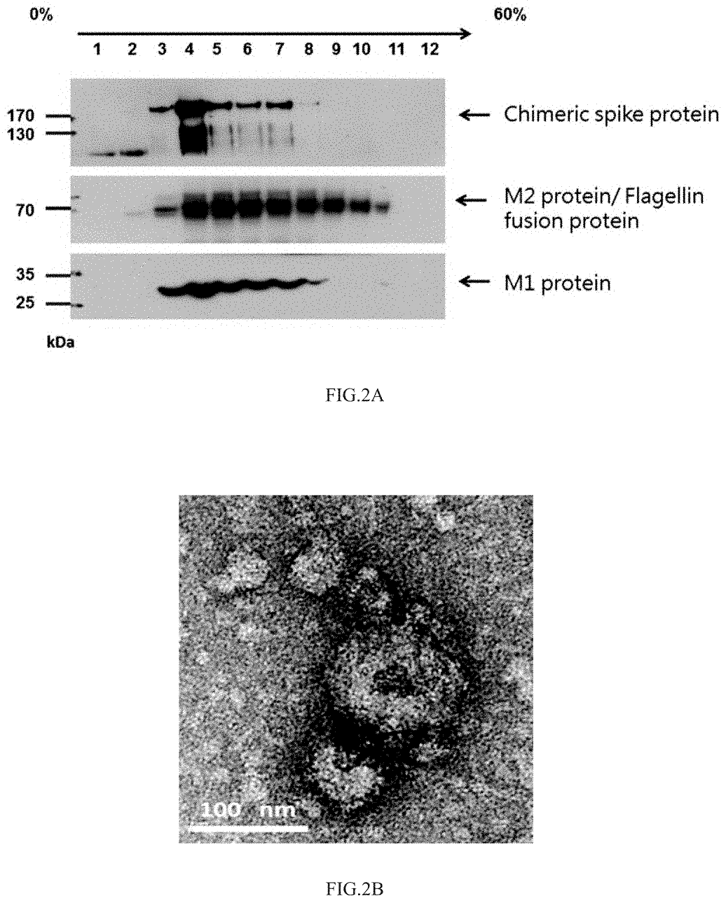

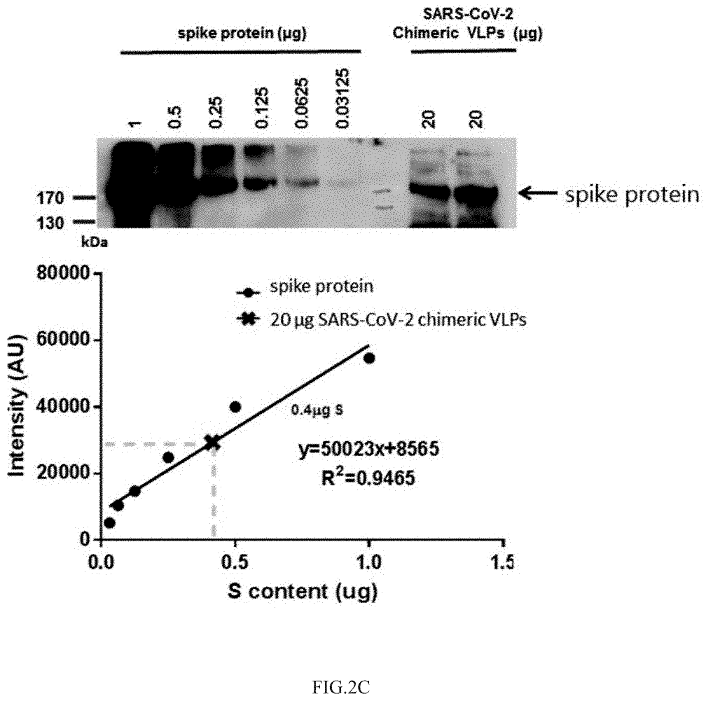

The detailed analysis methods of SDS-PAGE and western blotting of samples from sucrose gradient purification are as follows: first, 15 μL of samples from each sucrose gradient is taken and mixed with 5 μL loading buffer solution containing SDS, and then is boiled for 10 minutes. The samples are then loaded into an SDS-PAGE gel (10% separating gel), and are performed at 80V for 20 minutes. After the proteins of each sample are transferred from the stacking gel to the separating gel, the voltage is adjusted to 140V for about 2.5-3 hours to separate proteins of different sizes. After the electrophoresis, western blotting is performed to confirm the sucrose gradient solution containing the chimeric spike protein, the M1 protein, and the M2/flagellin protein. First, the SDS-PAGE gel is placed in the transfer tank with an NC membrane to transfer proteins at 135V for 40 minutes. Anti-spike protein antibody (GTX135356; Genetax; the dilution ratio is 1:1000), anti-M1 protein antibody (GTX127356; Genetax; the dilution ratio is 1:2000), and anti-M2 protein antibody (GTX125951; Genetax; the dilution ratio is 1:3000) are used as primary antibodies, and anti-rabbit IgG-HRP antibody (dilution ratio is 1:10000) is used as secondary antibody. The operation of western blotting is performed by the aforementioned method. The results are shown as FIG. 2A. Since the sizes of the chimeric spike protein, the M1 protein, and the M2 protein/flagellin fusion protein are about 180 kDa, 28 kDa, and 70 kDa, respectively, the virus-like particles of the present invention might locate in the sucrose gradient ranging from 20% to 40% where exhibit the protein signals with the targeted protein sizes.

In order to further confirm the size and shape of the SARS-CoV-2 chimeric VLP of the present invention in the sucrose gradient No. 3, No. 4, No. 5, No. 6, and No. 7 solutions, a transmission electron microscope (TEM) is used to confirm the appearance of the virus-like particle. The result is shown in FIG. 2B that the size of the virus-like particles of the present invention is about 100 nanometers in diameter, which is similar to the size of influenza virus particles, and spike protein structure can be obviously observed on the periphery of the virus-like particles. Therefore, with the method described in the embodiment, the SARS-CoV-2 chimeric VLP of the present invention can be obtained. The following would use the virus-like particles as a vaccine for efficacy testing.

In order to further evaluate the content of the spike protein in the SARS-CoV-2 chimeric VLP of the present invention, the western blotting is used to analyze serially diluted spike protein standards (purchased from Sino biological, catalog number 40589-V08B1, starting concentration of 1 μg, 2 times dilution), and ImageJ software is then used to analyze the bend area of each dilution concentration on the film to draw a linear equation as a standard curve. The content of the spike protein in the SARS-CoV-2 chimeric VLP of the present invention is also carried out by the western blotting and quantified by ImageJ software, and then the bend area on the film is plotted against the known concentrations and drew a linear regression to obtain the weight of the spike protein contained on the virus-like particles. The result is shown in FIG. 2C that 20 μg of the SARS-CoV-2 chimeric VLP of the present invention contains 0.4 μg of spike protein, which is about 2% of the total weight of the SARS-CoV-2 chimeric VLP.

### Example 2

### Systemic Immunogenic Effects of the SARS-CoV-2 Chimeric VLP in Mice

One embodiment of the present invention is to examine whether the SARS-CoV-2 chimeric VLP of the present invention can indeed induce the production of antigen-specific antibodies without additional adjuvants; first, the virus-like particles are injected into mice, and after a period of time, the sera are collected and the antibodies against SARS-CoV-2 are analyzed.

First, the SARS-CoV-2 chimeric VLP of the present invention, alone or in combination with an adjuvant, is prepared into a 100 μL vaccine solution using phosphate buffered saline solution (dissolving 137 mM sodium chloride, 2.7 mM potassium chloride, 4.3 mM disodium hydrogen phosphate, and 1.4 mM potassium dihydrogen phosphate in deionized water, pH 7.4; hereafter referred to as PBS solution); wherein, alum and/or monophosphoryl lipid A (MPLA) is used as adjuvants in the embodiment, and the following 13 groups are respectively prepared: (1) control group only containing PBS solution, (2) experimental group containing 2 μg of the SARS-CoV-2 chimeric VLP of the present invention (2 μg S FliC-cVLP), (3) comparison group containing 2 μg of the SARS-CoV-2 chimeric VLP of the present invention and 300 μg of alum (2 μg S FliC-cVLP+Alum), (4) comparison group containing 2 μg of the SARS-CoV-2 chimeric VLP of the present invention and 30 μg of monophosphoryl lipid A (2 μg S FliC-cVLP+MPLA), (5) comparison group containing 2 μg of the SARS-CoV-2 chimeric VLP of the present invention, 300 μg of alum, and 30 μg of monophosphoryl lipid A (2 μg S FliC-cVLP+Alum+MPLA), (6) experimental group containing 20 μg of the SARS-CoV-2 chimeric VLP of the present invention (20 μg S FliC-cVLP), (7) comparison group containing 20 μg of the SARS-CoV-2 chimeric VLP of the present invention and 300 μg of alum (20 μg S FliC-cVLP+Alum), (8) comparison group containing 20 μg of the SARS-CoV-2 chimeric VLP of the present invention and 30 μg of monophosphoryl lipid A (20 μg S FliC-cVLP+MPLA), (9) comparison group containing 20 μg of the SARS-CoV-2 chimeric VLP of the present invention, 300 μg of alum, and 30 μg of monophosphoryl lipid A (20 μg S FliC-cVLP+Alum+MPLA), (10) experimental group containing 40 μg of the SARS-CoV-2 chimeric VLP of the present invention (40 μg S FliC-cVLP), (11) comparison group containing 40 μg of the SARS-CoV-2 chimeric VLP of the present invention and 30 μg of monophosphoryl lipid A (40 μg S FliC-cVLP+MPLA), (12) experimental group containing 80 μg of the SARS-CoV-2 chimeric VLP of the present invention (80 μg S FliC-cVLP), (13) comparison group containing 80 μg of the SARS-CoV-2 chimeric VLP of the present invention and 30 μg of monophosphoryl lipid A (80 μg S FliC-cVLP+MPLA). Next, female BALB/c mice (6 to 8 weeks old) are intramuscularly injected with each of the 13 groups of solutions at the zeroth week and the third week, respectively. Blood samples of each group of mice are collected two weeks after the second immunization, and the serum is collected by centrifuging at 3000 rpm for 15 minutes, and then stored at −20° C. for further examination. All experiments are conducted in accordance with the guidelines of the Laboratory Animal Center of the National Tsing Hua University (NTHU). Animal use protocols are reviewed and approved by the NTHU Institutional Animal Care and Use Committee (approval no. 109047).

Next, to assess whether SARS-CoV-2 chimeric VLP effectively elicits the systemic immune responses, enzyme-linked immunosorbent assay (ELISA) is used to determine the antibody titers in mouse serum. First, recombinant spike protein (catalog number 40589-V08B1) and receptor binding domain protein (RBD, catalog number 40592-V08H) proteins are obtained from Sino Biological Inc., and are coated onto two different 96-well plates at 1 μg/mL and 2 μg/mL respectively in 0.05 M carbonic acid buffer (i.e. coating buffer) overnight at 4° C. Coating buffers are aspirated and each well is washed with PBS solution containing 0.05% Tween 20 (hereinafter referred to as PBST solution) three times to wash off excess recombinant spike protein or RBD protein. Each well is blocked with 150 μL blocking buffer (1% bovine serum albumin (BSA) in PBST solution) at room temperature for 2 hours to avoid non-specific binding. Blocking buffers are aspirated and each well is washed with PBST solution three times to wash off excess blocking buffer. Next, the serum of each group of mice is pre-diluted 1:50, followed by a serial dilution in dilution buffer (0.05% tween 20 and 1% BSA in PBST solution). The diluted serum are added into the 96-well culture plate at room temperature for 1 hour to allow the specific antibodies in the serum to bind to the spike protein or RBD protein. The serial dilutions are aspirated and each well is washed with PBST solution three times to wash off excess serum. Following this, 100 μL of horseradish peroxidase (HRP) conjugated anti-mouse IgG antibody (goat anti-mouse IgG antibody is used in the example, the dilution ratio is 1:30000) is added into each well and incubated at room temperature in the dark for 1 hours. The solution in each well is aspirated and each well is washed with PBST solution three times to wash off excess antibodies. Finally, 100 μL of the substrate 3,3′,5,5′-Tetramethylbenzidine (TMB, purchased from BioLegend) of HRP is added to each well and incubated in the dark for 15 min. The reaction is stopped with 100 μL of 2 N H₂SO₄. The optical density at 450 nm is then measured using an ELISA analyzer (DYNEX MRX II). The end-point titration values were calculated by a final serial dilution higher than 0.2 in the optical density value.

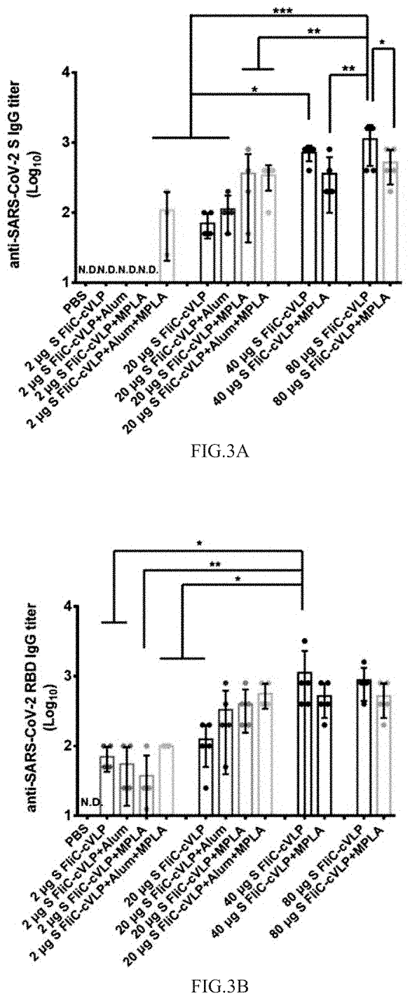

The titers of spike-specific and RBD-specific antibodies in the serum of mice injected with the SARS-CoV-2 chimeric VLP of the present invention alone or in combination with an adjuvant is shown as FIG. 3A and FIG. 3B, respectively. As shown in FIG. 3A, after immunization, the titer of anti-spike protein antibody in mouse serum would increase with the increase of the injected dose of the virus-like particles, and compared with the groups with additional adjuvant, the high dose (i.e. dose of 40 μg or 80 μg) of the SARS-CoV-2 chimeric VLP of the present invention can effectively induce higher levels of spike-specific antibodies against SARS-CoV-2 without additional adjuvants. As shown in FIG. 3B, the titer of anti-RBD antibody also increases with the increase of the injected dose of the virus-like particles, and compared with the groups with additional adjuvant, the high dose (i.e. dose of 40 μg or 80 μg) of the SARS-CoV-2 chimeric VLP of the present invention can effectively induce higher titer antibody responses to RBD protein without additional adjuvants.

These results indicate that the SARS-CoV-2 chimeric VLP of the present invention can indeed effectively induce an immune response against SARS-CoV-2, especially spike protein and RBD protein thereof. In addition, compared with the prior art that requires additional adjuvants for immune induction, the SARS-CoV-2 chimeric VLP of the present invention which self-carry flagellin has an unexpectedly excellent effect that can induce individuals to produce higher antibody titers. Therefore, the SARS-CoV-2 chimeric VLP of the present invention can indeed effectively induce the production of antibodies against SARS-CoV-2, and have the potential to be used as a vaccine for SARS-CoV-2.

### Example 3

### The SARS-CoV-2 Chimeric VLP Tends to Induce the Immune Response of Type 1 Helper T Cells

Vaccines need to activate helper T cells while inducing individual immunity to produce antibodies, and helper T cells can be divided into the type I helper T cells (Th1) and the type II helper T cells (Th2). Studies have shown that if the vaccine tends to activate type II helper T cells, the immune system is prone to induce adverse effects such as allergies. Therefore, it is necessary to evaluate the bias of type I and type II helper T cells in the immune response.

Herein, in one embodiment of the present invention, the SARS-CoV-2 chimeric VLP of the present invention are further examined for the tendency thereof to induce type I helper T cells or type II helper T cells; wherein, IgG2a and IgG1 are known to be the index antibodies of the type I helper T cells and the type II helper T cells, respectively; and IFN-γ and IL-5 are important cytokines that promotes type I helper T cells and the type II helper T cells, respectively; therefore, in the example, ELISA is used to detect the amount of the two indicator antibodies produced in the mice serum after being injected with the SARS-CoV-2 chimeric VLP of the present invention, and the amount of IFN-γ and IL-5 released by the spleen cells of these mice.

First, in the manner described in Example 2, the vaccine solutions of the SARS-CoV-2 chimeric VLP of the present invention are prepared, and the following 13 groups are also respectively prepared: (1) control group only containing PBS solution, (2) experimental group containing 2 μg of the SARS-CoV-2 chimeric VLP of the present invention (2 μg S FliC-cVLP), (3) comparison group containing 2 μg of the SARS-CoV-2 chimeric VLP of the present invention and 300 μg of alum (2 μg S FliC-cVLP+Alum), (4) comparison group containing 2 μg of the SARS-CoV-2 chimeric VLP of the present invention and 30 μg of monophosphoryl lipid A (2 μg S FliC-cVLP+MPLA), (5) comparison group containing 2 μg of the SARS-CoV-2 chimeric VLP of the present invention, 300 μg of alum, and 30 μg of monophosphoryl lipid A (2 μg S FliC-cVLP+Alum+MPLA), (6) experimental group containing 20 μg of the SARS-CoV-2 chimeric VLP of the present invention (20 μg S FliC-cVLP), (7) comparison group containing 20 μg of the SARS-CoV-2 chimeric VLP of the present invention and 300 μg of alum (20 μg S FliC-cVLP+Alum), (8) comparison group containing 20 μg of the SARS-CoV-2 chimeric VLP of the present invention and 30 μg of monophosphoryl lipid A (20 μg S FliC-cVLP+MPLA), (9) comparison group containing 20 μg of the SARS-CoV-2 chimeric VLP of the present invention, 300 μg of alum, and 30 μg of monophosphoryl lipid A (20 μg S FliC-cVLP+Alum+MPLA), (10) experimental group containing 40 μg of the SARS-CoV-2 chimeric VLP of the present invention (40 μg S FliC-cVLP), (11) comparison group containing 40 μg of the SARS-CoV-2 chimeric VLP of the present invention and 30 μg of monophosphoryl lipid A (40 μg S FliC-cVLP+MPLA), (12) experimental group containing 80 μg of the SARS-CoV-2 chimeric VLP of the present invention (80 μg S FliC-cVLP), (13) comparison group containing 80 μg of the SARS-CoV-2 chimeric VLP of the present invention and 30 μg of monophosphoryl lipid A (80 μg S FliC-cVLP+MPLA). Next, the mice are immunized and serum samples from each group of mice are collected in the manner described in Example 2, and the mice are sacrificed to collect spleen cells of each group of mice. After the spleen cells are grinded, the grinded spleen cells are filtered into centrifuge tubes by a cell strainer, and then centrifuged at 1500 rpm for 5 minutes. The supernatant is then removed and 4 mL of red blood cell lysate is added and then stand for 5 minutes at room temperature. 36 mL of RPMI cell culture medium (purchased from Thermo Scientific™, USA) is then added to stop reactions of the red blood cell lysate, and centrifuged at 1500 rpm for 5 minutes. After cell number is counted, the spleen cells are then cultured in a 96-well plate at an amount of 5×10⁶ cells/well, and 5 μg of spike protein (purchased from Sino Biological Company, catalog number 40589-V08B1) is added for 72 hours under the condition of 5% carbon dioxide at 37° C. to stimulate the spleen cells.

The serum samples collected from each group of mice are used to determine the titer of IgG2a antibody and IgG1 antibody by ELISA. First, recombinant spike protein (catalog number 40589-V08B1) is obtained from Sino Biological Inc., and is coated onto two different 96-well plates at 1 g/mL in coating buffer overnight at 4° C. Coating buffers are aspirated and each well is washed with PBST solution three times to wash off excess recombinant spike protein. Each well is blocked with 150 μL blocking buffer at room temperature for 2 hours to avoid non-specific binding. Blocking buffers are aspirated and each well is washed with PBST solution three times to wash off excess blocking buffer. Next, the serum of each group of mice is pre-diluted 1:50, followed by a serial dilution in dilution buffer. The serial dilutions of the serum are added into the 96-well culture plate at room temperature for 1 hour to allow the specific antibodies in the serum to bind to the spike protein. The serial dilutions are aspirated and each well is washed with PBST solution three times to wash off excess serum. Following this, 100 μL of HRP conjugated anti-mouse IgG1 antibody and HRP conjugated anti-mouse IgG2a antibody (goat anti-mouse IgG1 antibody and goat anti-mouse IgG2a antibody is used in the example, the dilution ratio is 1:50000) is added into each well at room temperature in the dark for 1 hours. The solution in each well is aspirated and each well is washed with PBST solution three times to wash off excess antibodies. Finally, 100 μL of the substrate TMB of HRP is added to each well and incubated in the dark for 15 min. The reaction is stopped with 100 μL of 2 N H₂SO₄. The optical density at 450 nm is then measured using an ELISA analyzer (DYNEX MRX II). The end-point titration values were calculated by a final serial dilution higher than 0.2 in the optical density value.

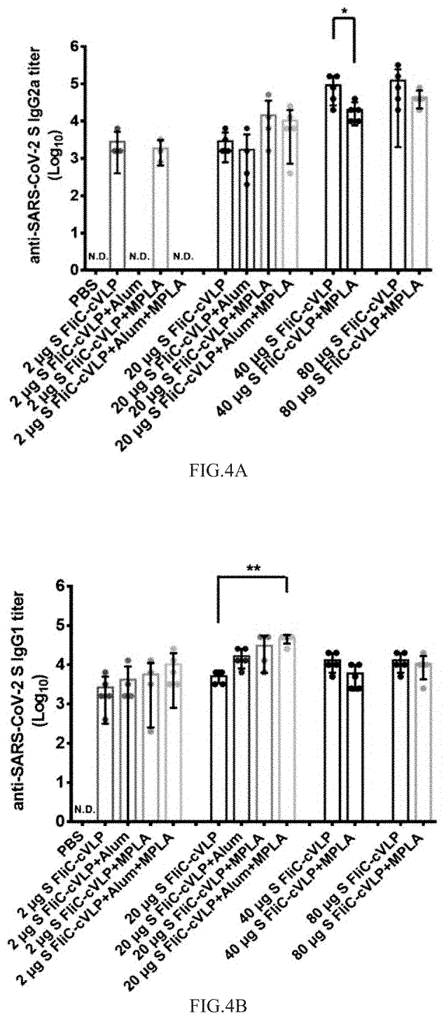

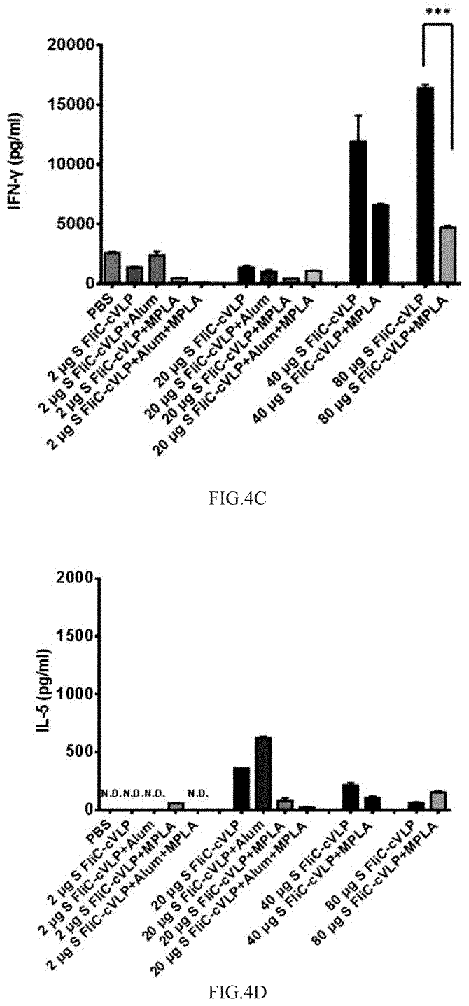

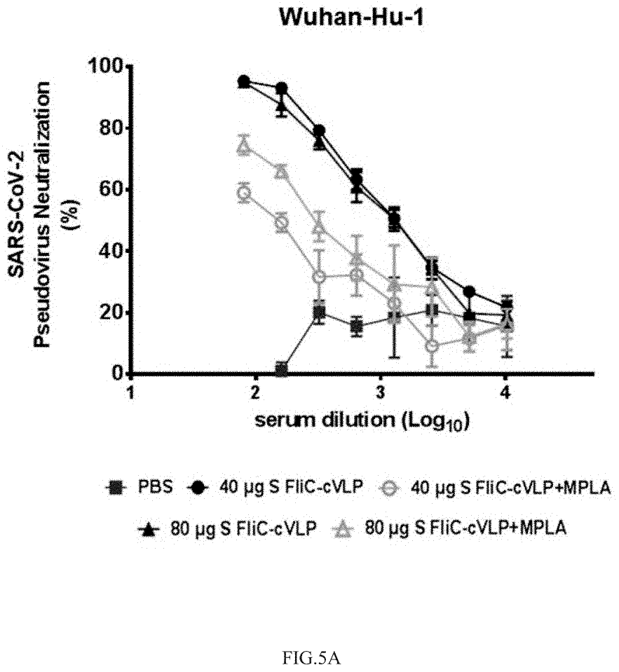

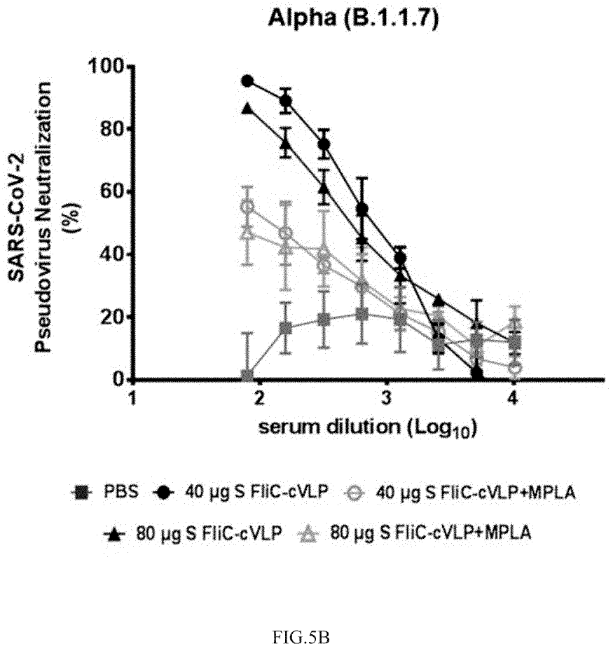

The titers of the anti-spike protein IgG2a antibody and the anti-spike protein IgG1 antibody in the serum of mice injected with the SARS-CoV-2 chimeric VLP of the present invention is shown as FIG. 4A and FIG. 4B, respectively. As shown in FIG. 4A, after immunization injection, the titer of anti-spike protein IgG2a antibody in mouse serum would increase with the increase of the injected dose of the virus-like particles, and compared with the groups with additional adjuvant, the high dose (i.e. dose of 40 μg or 80 μg) of the SARS-CoV-2 chimeric VLP of the present invention can effectively induce higher titer of the IgG2a antibody against spike protein of SARS-CoV-2 without additional adjuvants. As shown in FIG. 4B, no matter the dose of the immunization injection is high or low, or there is an additional adjuvant or not, after being immunized with the SARS-CoV-2 chimeric VLP of the present invention, the titers of the IgG1 antibody against the spike protein in the mouse serum are not significantly different.

The results indicate that the SARS-CoV-2 chimeric VLP of the present invention tend to induce the immune response of the type I helper T cells; therefore, the adverse immune system side effects such as allergies caused by the immune response of the type II helper T cells can be reduced. In addition, compared with the prior art that requires additional adjuvants for immune induction, the SARS-CoV-2 chimeric VLP of the present invention which self-carry flagellin has an unexpectedly excellent effect that can induce individuals to produce higher IgG2a antibody titers.

The spleen cells collected from each group of mice are detected by ELISA for the concentration of IFN-γ. The following procedures for detecting the concentration of IFN-γ refer to the manufacturer's instructions (BioLegend, 430801). First, IFN-γ capture antibodies are coated onto two different 96-well plates at the concentration of a 200-fold dilution of the original tube in coating buffer overnight at 4° C. Coating buffers are aspirated and each well is washed with PBST solution three times to wash off excess IFN-γ capture antibodies. Each well is blocked with 150 μL blocking buffer at room temperature for 2 hours to avoid non-specific binding. Blocking buffers are aspirated and each well is washed with PBST solution three times to wash off excess blocking buffer. Next, the spleen cells of each group of mice are pre-diluted 2 folds, followed by a serial dilution in dilution buffer. The serial dilutions of the serum are added into the 96-well culture plate at room temperature for 1 hour to allow the specific antibodies in the serum to bind to the IFN-γ capture antibodies. The serial dilutions are aspirated and each well is washed with PBST solution four times to wash off excess serum. Following this, IFN-γ detection antibodies at a 200-fold dilution of the original tube are added into each well for 1 hour. The IFN-γ detection antibodies are aspirated and each well is washed with PBST solution to wash off excess IFN-γ detection antibody. Avidin at a 1000-fold dilution of the original tube is then added into each well for 0.5 hour, and then each well is washed with PBST five times at intervals 30 seconds. Finally, 100 μL of the substrate TMB of HRP is added to each well and incubated in the dark for 15 min. The reaction is stopped with 100 μL of 2 N H₂SO₄. The optical density at 450 nm is then measured using an ELISA analyzer (DYNEX MRX II). The standard curve is used to calculate the concentration of IFN-γ in the spleen cells of each group of mice.

After vaccinated with the SARS-CoV-2 chimeric VLP of the present invention, the concentration of IFN-γ in the spleen cells of mice, which is then stimulated with spike protein, is shown as FIG. 4C. As shown in FIG. 4C, the trend of the concentration of IFN-γ are consistent with the trend of the titer of the anti-spike protein IgG2a antibody. That is, the concentration of IFN-γ in mouse serum would increase with the increase of the injected dose of the virus-like particles, and compared with the groups with additional adjuvant, the high dose (i.e. dose of 40 μg or 80 μg) of the SARS-CoV-2 chimeric VLP of the present invention can effectively induce higher concentration of IFN-γ without additional adjuvants.

The spleen cells collected from each group of mice are detected by ELISA for the concentration of IL-5. The following procedures for detecting the concentration of IL-5 refer to the manufacturer's instructions (BioLegend, 430801). First, IL-5 capture antibodies are coated onto two different 96-well plates at the concentration of a 200-fold dilution of the original tube in coating buffer overnight at 4° C. Coating buffers are aspirated and each well is washed with PBST solution three times to wash off excess IL-5 capture antibodies. Each well is blocked with 150 μL blocking buffer at room temperature for 2 hours to avoid non-specific binding. Blocking buffers are aspirated and each well is washed with PBST solution three times to wash off excess blocking buffer. Next, the spleen cells of each group of mice are pre-diluted 2 folds, followed by a serial dilution in dilution buffer. The serial dilutions of the serum are added into the 96-well culture plate at room temperature for 1 hour to allow the specific antibodies in the serum to bind to the IL-5 capture antibodies. The serial dilutions are aspirated and each well is washed with PBST solution four times to wash off excess serum. Following this, IL-5 detection antibodies at a 200-fold dilution of the original tube are added into each well for 1 hour. The IL-5 detection antibodies are aspirated and each well is washed with PBST solution to wash off excess IL-5 detection antibody. Avidin at a 1000-fold dilution of the original tube is then added into each well for 0.5 hour, and then each well is washed with PBST five times at intervals 30 seconds. Finally, 100 μL of the substrate TMB of HRP is added to each well and incubated in the dark for 15 min. The reaction is stopped with 100 μL of 2 N H₂SO₄. The optical density at 450 nm is then measured using an ELISA analyzer (DYNEX MRX II). The standard curve is used to calculate the concentration of IL-5 in the spleen cells of each group of mice.

After vaccinated with the SARS-CoV-2 chimeric VLP of the present invention, the concentration of IL-5 in the spleen cells of mice, which is then stimulated with spike protein, is shown as FIG. 4D. As shown in FIG. 4D, no matter the injected dose is high or low, or with or without additional adjuvants, the concentration of IL-5 in the spleen cells of mice in each group is extremely weak and there is not much difference between the groups, indicating that the SARS-CoV-2 chimeric VLP of the present invention do not induce the immune response of the type II helper T cells.

According to the results, it is more certain that the SARS-CoV-2 chimeric VLP of the present invention can stimulate the secretion of more IFN-γ, and then induce a stronger immune response of the type I helper T cells. In addition, compared with the prior art that requires additional adjuvants for immune induction, the SARS-CoV-2 chimeric VLP of the present invention which self-carry flagellin has an unexpectedly excellent effect that can induce individuals to produce higher concentrations of IFN-γ.

### Example 4

### Induction of Neutralizing Antibodies Against SARS-CoV-2 by SARS-CoV-2 Chimeric VLP

In one embodiment of the present invention, the efficacy of the SARS-CoV-2 chimeric VLP of the present invention to induce immunized mice to produce neutralizing antibodies against the ancestral Wuhan-Hu-1 strain, the Alpha (B.1.1.7, British) variant, the Beta (B.1351, South African) variant, and the Delta (B.1.617.2, Indian) variant of SARS-CoV-2 is assessed by pseudovirus neutralization assay.

First, in the manner described in Example 2, the vaccine solutions of the SARS-CoV-2 chimeric VLP of the present invention are prepared, and the following 5 groups are also respectively prepared: (1) control group only containing PBS solution, (2) experimental group containing 40 μg of the SARS-CoV-2 chimeric VLP of the present invention (40 μg S FliC-cVLP), (3) comparison group containing 40 μg of the SARS-CoV-2 chimeric VLP of the present invention and 30 μg of monophosphoryl lipid A (40 μg S FliC-cVLP+MPLA), (4) experimental group containing 80 μg of the SARS-CoV-2 chimeric VLP of the present invention (80 μg S FliC-cVLP), (5) comparison group containing 80 μg of the SARS-CoV-2 chimeric VLP of the present invention and 30 μg of monophosphoryl lipid A (80 μg S FliC-cVLP+MPLA). Next, the mice are immunized and serum samples from each group of mice are collected in the manner described in Example 2 for the following tests.

One day before determining the titers of neutralizing antibodies against SARS-CoV-2, HEK-293T cells stably expressing human angiotensin-converting enzyme 2 (hACE2) are planted in each well of a 96-well culture plate at 37° C. for one day; wherein, DMEM (Dulbecco's Modified Eagle Medium) containing 1% fetal bovine serum (FBS) is used as the cell culture medium. The serum samples are serially diluted from 80-fold and respectively incubated with 1,000 TU (transducing units) of the ancestral Wuhan-Hu-1 strain, the Alpha variant strain, the Beta variant strain, and the Delta variant strain of SARS-CoV-2 pseudotyped lentivirus for 1 hour at 37° C. The mixture is then added into the aforementioned 96-well culture plate to infect the HEK-293T cells at 37° C. for 16 hours. The culture medium is replaced with fresh complete DMEM containing 10% FBS at 16 hours post-infection and cells are continuously cultured for another 48 hours before being subjected to luciferase assay (Promega Bright-Glo™ Luciferase Assay System). The percentage of inhibition is calculated as the ratio of the loss of luciferase readout (RLU) in the presence of serum to that of no serum control, and the calculation formula used for the calculation was (RLU Control−RLU Serum)/RLU Control; wherein, in order to produce the pseudotyped lentivirus of each SARS-CoV-2 strain, the pLAS2W.FLuc.Ppur reporter plasmid of the firefly luciferase, pcDNA3.1(+) plasmid expressing full-length spike protein gene of SARS-CoV-2 (Wuhan-Hu-1, B.1.1.7, B.1.351 or B.1.617.2), and HIV gag-pol plasmid (pCMVΔR8.91) are all co-transfected into HEK293T cells by TransIT-LT1 transfection reagent (Purchased from Mirus Bio), and the medium is collected and concentrated 48 hours after transfection, and then by detecting the luciferase activity of SARS-CoV-2-Spp transcription to evaluate the titer of lentivirus.

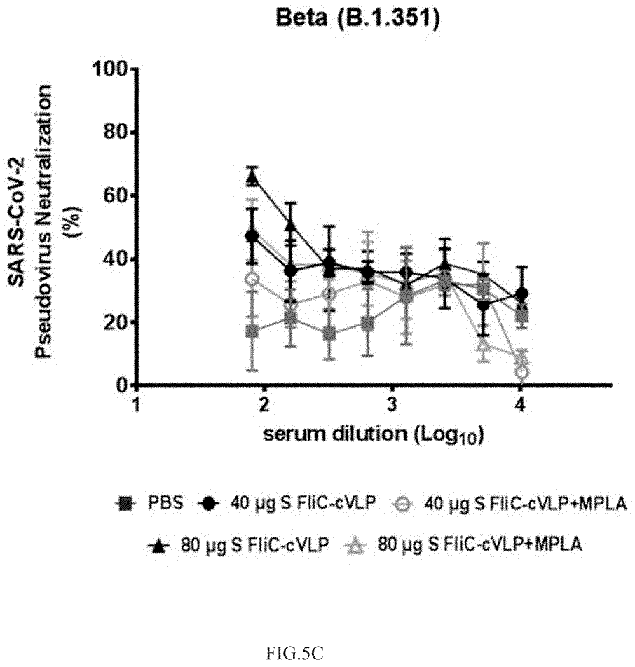

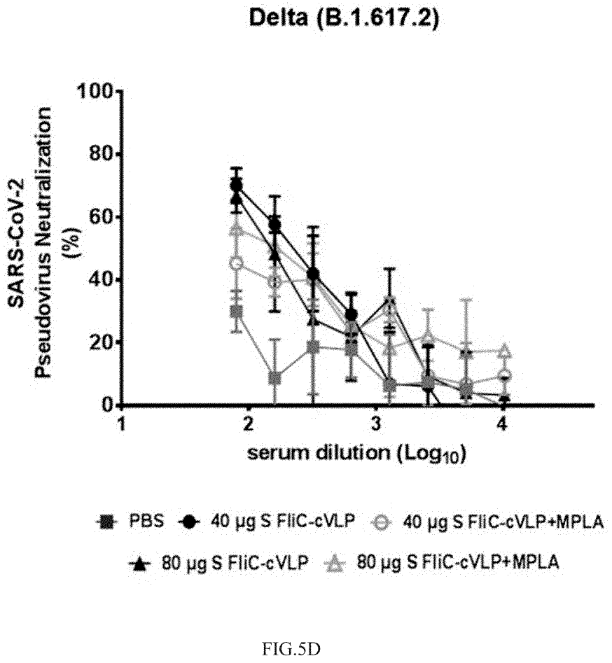

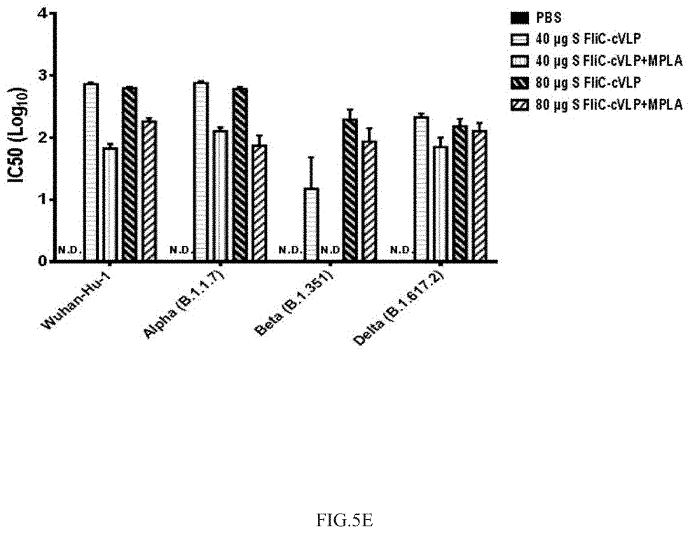

The neutralizing result of the serum of mice immunized with the SARS-CoV-2 chimeric VLP of the present invention against the ancestral Wuhan-Hu-1 strain of SARS-CoV-2 by pseudotyped lentivirus neutralization assay is shown in FIG. 5A, the neutralizing result of the serum of mice against the Alpha variant strain of SARS-CoV-2 is shown in FIG. 5B, the neutralizing result of the serum of mice against the Beta variant strain of SARS-CoV-2 is shown in FIG. 5C, the neutralizing result of the serum of mice against the Delta variant strain of SARS-CoV-2 is shown in FIG. 5D, and the IC50 of the four neutralizing results is shown in FIG. 5E.

As shown in FIGS. 5A to 5E, the neutralizations of serum of mice, which is immunized with the SARS-CoV-2 chimeric VLP of the present invention with a high dose (i.e., dose of 40 μg or 80 μg), to the ancestral Wuhan-Hu-1 strain, the Alpha variant strain, the Beta variant strain, and the Delta variant strain of SARS-CoV-2 are significantly higher than that of the groups combined with additional adjuvants. The result indicates that the SARS-CoV-2 chimeric VLP of the present can induce high-titer neutralizing antibodies against SARS-CoV-2 without additional adjuvants, and have excellent efficacy not only for the ancestral Wuhan-Hu-1 strain of SARS-CoV-2, but also for the current three variant strains of SARS-CoV-2.

### Example 5

### The Design and Construction of the Vector Expressing the Chimeric Bivalent Virus-Like Particle of SARS-CoV-2 and Influenza Virus

One embodiment of the present invention is to construct a virus-like particle that can simultaneously produce immunity to SARS-CoV-2 and influenza virus. Therefore, based on the vector constructed in Example 1, the hemagglutinin influenza virus (SEQ ID NO: 9) are cloned on pFastBac™ Dual vector (purchased from Invitrogen, USA) between the restriction enzyme cutting sites of BamHI and NotI, and the Bac-to-Bac® baculovirus expression system described in Example 1 is used to make the other recombinant baculovirus. Next, the recombinant baculovirus and the recombinant baculovirus of Example 1 are co-infected with Sf9 cells under the 2:1 MOI (multiplicity of infection) for five days, and then, the methods described in Example 1 are used to collect, concentrate, and use a discontinuous sucrose gradient to purify the virus-like particles of the present invention.

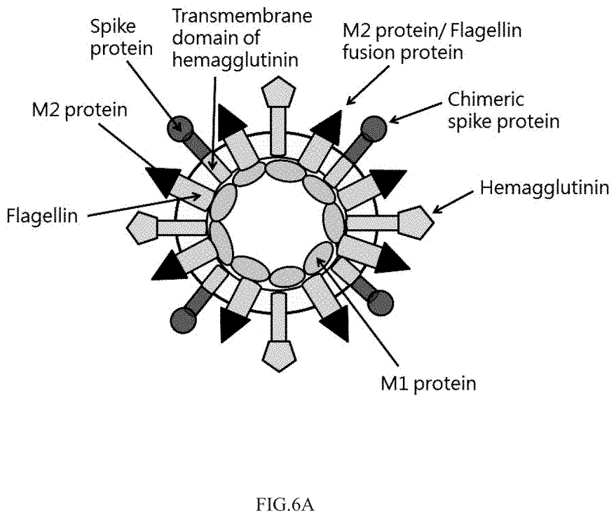

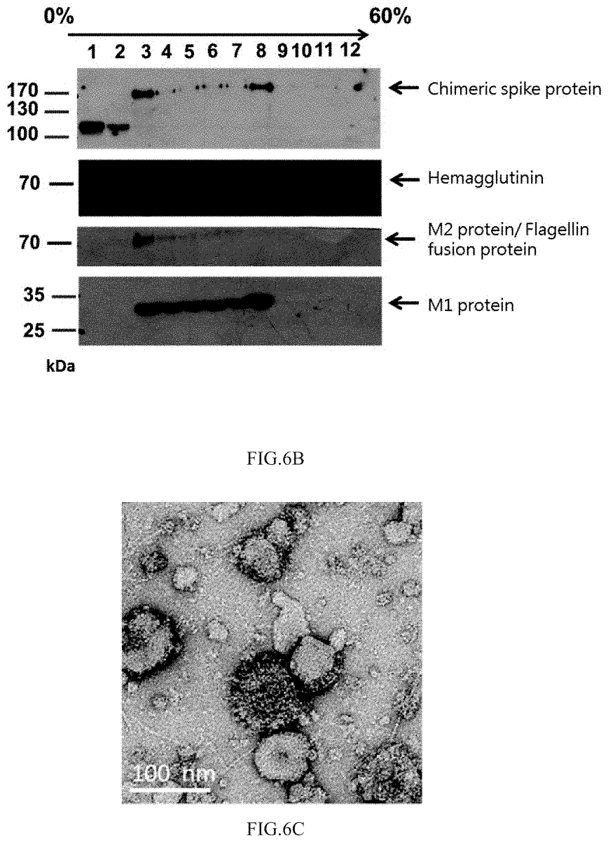

As shown in FIG. 6A, the M1 protein and the M2 protein are used in the present invention to form a virus-like particle skeleton, which the M1 protein is located on the inner side and the M2 protein is located on the outer side to form a spherical structure, and the chimeric spike protein is expressed on the surface of the virus-like particle skeleton, and the flagellin is fused to the outer side of the M2 protein.

The methods of SDS-PAGE and western blotting described in Example 1 are used to analyze the samples from sucrose gradient purification. As shown in FIG. 6B, the virus-like particles of the present invention should be located in the sucrose gradient No. 3 and No. 8 solutions which are simultaneously detected with protein signals of chimeric spike protein, M1 protein, M2 protein/flagellin fusion protein, and hemagglutinin.

The transmission electron microscope is further used to confirm the appearance of the SARS-CoV-2 and influenza virus virus-like particle in the sucrose gradient No. 3 and No. 8 solutions. The result is shown in FIG. 6C that the size of the virus-like particles of the present invention is about 60-100 nanometers in diameter, which is similar to the size of influenza virus particles, and spike protein structure can be obviously observed on the periphery of the virus-like particles. Therefore, with the method described in the embodiment, the chimeric SARS-CoV-2 and influenza virus virus-like particles of the present invention can be obtained.

In summary, the SARS-CoV-2 chimeric VLP of the present invention is successfully assembled into a virus-like particle expressing a spike protein, M1 protein, and M2 protein/flagellin fusion protein constructed on a single vector of the present invention. The SARS-CoV-2 chimeric VLP of the present invention can effectively elicit the production of antibodies against SARS-CoV-2 without additional adjuvants in vaccinated individuals. Further analyses show that the amount of serum IgG2a antibodies against spike protein and the secretion of IFN-γ would increase accordingly as the dosage of the SARS-CoV-2 chimeric VLP of the present invention increases. The results indicate that the SARS-CoV-2 chimeric VLP of the present invention tends to elicit immune responses of the type I helper T cells, and could decrease the incidence of the adverse effects caused by the type II helper T cells. Furthermore, the SARS-CoV-2 chimeric VLP of the present invention can also produce high-titer neutralizing antibodies against not only the ancestral Wuhan-Hu-1 strain, but also the variant strains of Alpha (B.1.1.7, British), Beta (B.1351, South African), and Delta (B.1.617.2, Indian). As mentioned above, the SARS-CoV-2 chimeric VLP of the present invention can be effectively used as a vaccine against SARS-CoV-2.

In the SARS-CoV-2 chimeric VLP of the present invention, the transmembrane domain of the spike protein of SARS-CoV-2 is replaced with the transmembrane domain of the hemagglutinin of influenza virus to form a chimeric spike protein, which can be stably expressed on the surface of the virus-like particle skeleton. Because the SARS-CoV-2 chimeric VLP of the present invention is self-carrying flagellin proteins as an adjuvant, the SARS-CoV-2 chimeric VLP of the present invention can overcome the problem that the conventional virus-like particles still need to be used with an adjuvant.

Furthermore, in the vector for expressing the SARS-CoV-2 chimeric VLP of the present invention, all genes are under the control of their own promoter and terminator, so each of them can be expressed independently to reduce the risk of mutual interference. Moreover, different restriction enzyme cutting sites are designed on upstream and downstream of each gene above. The proteins exhibited on the SARS-CoV-2 chimeric VLP of the present invention can be easily replaced, accelerating the vaccine development when the evolving mutations of SARS-CoV-2 emerge. In this way, the present invention provides a new vaccine platform that does not require additional adjuvants and can be modified with antigens flexibly.

What is claimed is:

1. A severe acute respiratory syndrome coronavirus 2 (SARS-CoV-2) chimeric virus-like particle (VLP) vaccine composition, comprising a SARS-CoV-2 chimeric VLP, wherein, the SARS-CoV-2 chimeric VLP comprises: a virus-like particle skeleton, formed by a matrix protein 1 (M1 protein) of influenza virus and a matrix protein 2 (M2 protein) of influenza virus; and a chimeric spike protein of SARS-CoV-2, expressed on the surface of the virus-like particle skeleton, wherein the chimeric spike protein of SARS-CoV-2 is a spike protein of SARS-CoV-2 the transmembrane domain of which is replaced by a hemagglutinin (HA) of influenza virus.

2. The SARS-CoV-2 chimeric VLP vaccine composition according to claim 1, wherein an N-terminal end of the M2 protein of influenza virus is further connected to a flagellin.

3. The SARS-CoV-2 chimeric VLP vaccine composition according to claim 2, wherein a polyglycine linker is included between the M2 protein of influenza virus and the flagellin.

4. The SARS-CoV-2 chimeric VLP vaccine composition according to claim 1, wherein the SARS-CoV-2 chimeric VLP further comprises a HA of influenza virus expressed on the surface of the virus-like particle skeleton, forming a chimeric bivalent VLP of SARS-CoV-2 and influenza virus.

5. The SARS-CoV-2 chimeric VLP vaccine composition according to claim 4, wherein the chimeric bivalent VLP of SARS-CoV-2 and influenza virus is used as a bivalent vaccine composition against SARS-CoV-2 and influenza virus.

6. A method of the SARS-CoV-2 chimeric VLP vaccine composition according to claim 1 for eliciting an immune response against one or more SARS-CoV-2 variants infection, comprising immunizing a subject in need thereof a therapeutically effective amount of the SARS-CoV-2 chimeric VLP vaccine composition according to claim 1, which comprises a SARS-CoV-2 chimeric VLP, wherein the SARS-CoV-2 chimeric VLP comprises: a virus-like particle skeleton, formed by a M1 protein of influenza virus and a M2 protein of influenza virus; and a chimeric spike protein of SARS-CoV-2, expressed on the surface of the virus-like particle skeleton, the transmembrane domain of which is replaced by a HA of influenza virus.

7. The method according to claim 6, wherein the vaccine composition elicits an immune response of type I helper T cells.

8. The method according to claim 7, wherein the immune response of type I helper T cells includes a secretion of IgG2a, INF-γ, or the combination thereof.

9. The method according to claim 6, wherein the vaccine composition elicits high titer neutralizing antibodies against SARS-CoV-2.

10. The method according to claim 6, wherein the SARS-CoV-2 variants infection is caused by an ancestral Wuhan-Hu-1 strain, an Alpha (B.1.1.7, British) variant strain, a Beta (B.1351, South African) variant strain, a Delta (B.1.617.2, Indian) variant strain, and any combination thereof.

11. The method according to claim 6, wherein the SARS-CoV-2 chimeric VLP further comprises a HA of influenza virus expressed on the surface of the virus-like particle skeleton, forming a chimeric bivalent VLP of SARS-CoV-2 and influenza virus.

12. A recombinant vector expressing the SARS-CoV-2 chimeric VLP according to claim 1, comprising: a M1 protein gene of influenza virus, a M2 protein gene of influenza virus, and a chimeric spike protein gene of SARS-CoV-2; wherein the chimeric spike protein gene of SARS-CoV-2 is a spike protein gene of SARS-CoV-2 the transmembrane domain of which is replaced by a transmembrane domain of a HA of influenza virus.

13. The recombinant vector according to claim 12, wherein each of the M1 protein gene of influenza virus, the M2 protein gene of influenza virus, and the chimeric spike protein gene of SARS-CoV-2 is controlled by its own promoter and terminator and flanked by a pair of restriction enzyme cutting sites.

14. The recombinant vector according to claim 13, wherein a 5′-end of the M2 protein gene of influenza virus is connected to a flagellin gene.

15. The recombinant vector according to claim 12, wherein the recombinant vector further comprises a HA gene of influenza virus.
